(19) United States
(12) Patent Application Publication
(10) Pub. No.: US 2016/0339023 A1
(43) Pub. Date: Nov. 24, 2016

(54) Pharmaceutical Combinations

(71) Applicants: Fang Li, Arlington, MA (US); Hui-Qin WANG, Lexington, MA (US); Ensar HALILOVIC, Quincy, MA (US); Jinsheng LIANG, Shrewsbury, MA (US)

(72) Inventors: Fang Li, Arlington, MA (US); Hui-Qin WANG, Lexington, MA (US); Ensar HALILOVIC, Quincy, MA (US); Jinsheng LIANG, Shrewsbury, MA (US)

(21) Appl. No.: 15/107,232

(22) PCT Filed: Dec. 19, 2014
(86) PCT No.: PCT/IB2014/067139

§ 371 Date: Jun. 22, 2016

Related U.S. Application Data
(60) Provisional application No. 61/920,032, filed on Dec. 23, 2013.
(60) Provisional application No. 61/948,323, filed on Mar. 5, 2014.

(51) Int. Cl. A61K 31/506; A61K 31/496

(52) U.S. Cl. CPC A61K 31/506; A61K 31/496

(57) ABSTRACT

A pharmaceutical combination comprising (a) an ALK inhibitor, or a pharmaceutically acceptable salt thereof, and (b) at least one HDMA-2/p53 receptor inhibitor or a pharmaceutically acceptable salt, or at least one BRaf inhibitor or a pharmaceutically acceptable salt, and optionally a pharmaceutically acceptable carrier, for simultaneous, separate or sequential administration; the uses of such combination in the treatment of cancer; and methods of treating a subject suffering from a proliferative disease comprising administering a therapeutically effective amount of such combination.

### FIELD OF THE DISCLOSURE

The present disclosure relates to a pharmaceutical combination, e.g. a pharmaceutical product, comprising a combination of (i) a HDM-2/p53 inhibitor, or a pharmaceutically acceptable salt thereof, and (ii) an anaplastic lymphoma kinase (ALK) inhibitor, or a pharmaceutically acceptable salt thereof, which are jointly active in the treatment of proliferative diseases. In addition, the disclosure relates to a pharmaceutical combination, e.g. a pharmaceutical product, comprising (a) an anaplastic lymphoma kinase (ALK) inhibitor, or a pharmaceutically acceptable salt thereof, and (b) at least one BRaf inhibitor, or a pharmaceutically acceptable salt thereof. The disclosure also relates to corresponding pharmaceutical formulations, uses, methods, combinations and data carrier.

### BACKGROUND OF THE DISCLOSURE

Neuroblastoma is the most common cancer in infancy, accounting for 15% of all childhood cancer-related death. MYCN amplification is the major genetic aberration in high-risk neuroblastoma and is associated with poor outcome. Genome-wide association studies have identified activation mutations and high-level amplification of ALK in approximately 10% of neuroblastoma patients. In addition, ALK mutations can coexist with MYCN amplification, which defines a subset of ultra-high-risk neuroblastoma patients.

In addition, melanoma is a malignant neoplasm that arises in the pigmented portions of the skin and dermis. Therapy for melanoma includes a monotherapy with single molecule agents, such as crizotinib, which is described in International Patent Publication No. WO2007/105058. Approximately half of patients develop metastatic disease, typically to other organ systems, including but not limited to for example liver, lung and bone, and the incidence of new metastases continues to increase with time. The outcome for patients with metastatic disease is challenging, clinical therapeutic outcomes are poor and not currently optimal for a therapeutic perspective. No therapy for this disease has been approved to date.

In spite of numerous treatment options for patients with these exemplary types of cancer, there remains a need for effective and safe therapeutic agents and a need for new combination therapies that can be administered for the effective long-term treatment of cancer.

### SUMMARY OF THE DISCLOSURE

It has been unexpectedly discovered that the ALK inhibitor in combination with a HDM-2/p53 inhibitor can be a useful combination for treating proliferating disease, preferably cancer. Particularly, it was observed that ALK inhibitor 5-chloro-N2-(2-isopropoxy-5-methyl-4-(piperidin-4-yl)phenyl)-N4-[2-(propane-2-sulfonyl)-phenyl]pyrimidine-2,4-diamine (LDK378), in combination with a HDM-2/p53 inhibitor (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one (CGM097), promoted apoptosis in ALK mutant and p53 WT neuroblastoma cell lines. LDK378 inhibited ALK phosphorylation and CGM097 caused induction of p53 and its downstream target genes in these cell lines. For neuroblastomas, in contrast to the high frequency of p53 mutations observed in many human cancers, mutations of p53 have been reported in less than 2% of neuroblastomas. Wild-type (WT) p53 is required for the activation of p53 signaling by HDM-2/P53 inhibitors.

Further, for melanomas, it has now been found that a combination of the ALK inhibitor, for example LDK378, and a BRaf inhibitor, for example (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate (LGX818), is effective for the delay of progression or treatment of melanomas, metastatic melanomas, and mutant melanomas.

Therefore, it has now been surprisingly discovered that the combination of an effective amount of an ALK inhibitor, for example 5-chloro-N2-(2-isopropoxy-5-methyl-4-(piperidin-4-yl)phenyl)-N4-[2-(propane-2-sulfonyl)-phenyl]-pyrimidine-2,4-diamine, LDK378 (ceritinib), or a pharmaceutically acceptable salt thereof, and at least one HDM-2/p53 inhibitor or a pharmaceutically acceptable salt thereof; or at least one BRaf inhibitor, for example LGX818 results in unexpected improvement in the treatment of cancer, including but not limited to neuroblastomas, metastatic neuroblastomas, mutant neuroblastomas, melanomas, metastatic melanomas, and mutant melanomas.

When administered simultaneously, sequentially or separately, the pharmaceutical combinations disclosed herein inhibit cell proliferation and are surprisingly efficacious in neuroblastoma and melanoma models. For neuroblastomas, the therapeutic effect of the pharmaceutical combination is unexpectedly a synergistic interaction and completely inhibits the neuroblastoma as compared to a monotherapy with the ALK inhibitor alone, including but limited to LDK378, crizotinib or crizotinib resistant patients. It is expected that the anti-proliferative effect of the combination for treating melanomas is greater than the maximum effect that can be achieved with either type of therapeutic agent alone.

Specifically, the present disclosure provides the following aspects, advantageous features and specific embodiments, respectively alone or in combination, as listed in the following items:

1. A pharmaceutical combination comprising (i) a HDM-2/p53 inhibitor, or a pharmaceutically acceptable salt thereof, and (ii) an anaplastic lymphoma kinase (ALK) inhibitor, or a pharmaceutically acceptable salt thereof.
2. The pharmaceutical combination according to item 1, wherein the pharmaceutical combination comprises (i) a HDM-2/p53 inhibitor, or a pharmaceutically acceptable salt thereof, and (ii) an anaplastic lymphoma kinase (ALK) inhibitor, or a pharmaceutically acceptable salt thereof, separately or together.
3. The pharmaceutical combination according to item 1 or 2 for simultaneous or sequential use of the (i) a HDM-2/p53 inhibitor, or a pharmaceutically acceptable salt thereof, and (ii) an anaplastic lymphoma kinase (ALK) inhibitor, or a pharmaceutically acceptable salt thereof.
4. The pharmaceutical combination according to any one of items 1 to 3, further comprising at least one pharmaceutically acceptable carrier.
5. The pharmaceutical combination according to any one of items 1 to 4 in the form of a fixed combination.
6. The pharmaceutical combination according to any one of items 1 to 5 in the form of a kit of parts for the combined administration, wherein the HDM-2/p53 inhibitor, or a pharmaceutically acceptable salt thereof, and the anaplastic lymphoma kinase (ALK) inhibitor, or a pharmaceutically acceptable salt thereof, are administered jointly or independently at the same time or separately within time intervals.
7. The pharmaceutical combination according to any one of items 1 to 5 in the form of a pharmaceutical composition.
8. The pharmaceutical combination according to any one of items 1 to 7, wherein (i) a HDM-2/p53 inhibitor, or a pharmaceutically acceptable salt thereof, and (ii) an anaplastic lymphoma kinase (ALK) inhibitor, or a pharmaceutically acceptable salt thereof, are in a quantity which is jointly therapeutically effective for the treatment of cancer.
9. The pharmaceutical combination according to any one of items 1 to 8 in the form of a combination product or a pharmaceutical composition.
10. The pharmaceutical combination according to any one of items 1 to 9 for use as a medicine.
11. The pharmaceutical combination for use as a medicine according to item 10, wherein the HDM-2/p53 inhibitor, or a pharmaceutically acceptable salt thereof, is to be administered simultaneously or sequentially with an anaplastic lymphoma kinase (ALK) inhibitor, or a pharmaceutically acceptable salt thereof.
12. The pharmaceutical combination according to any one of items 1 to 9 for use in the treatment of cancer.
13. The pharmaceutical combination according to any one of items 1 to 9 for use in the treatment of cancer according to item 12, wherein the cancer comprises mutated anaplastic lymphoma kinase (ALK).
14. The pharmaceutical combination according to any one of items 1 to 9 for use in the treatment of cancer according to item 12 or 13, wherein the cancer is neuroblastoma or lung cancer, particularly wherein the cancer is neuroblastoma.
15. The pharmaceutical combination according to any one of items 1 to 9 for use in the treatment of cancer according to item 14, wherein the cancer is relapsed or high-risk neuroblastoma.
16. The pharmaceutical combination according to any one of items 1 to 9 for use in the treatment of cancer according to any one of items 12 to 15, wherein the cancer comprises functional p53 or is p53 wt.
17. The pharmaceutical combination according to any one of items 1 to 9 for use in the treatment of cancer according to any one of items 12 to 16, wherein the cancer is in a pediatric patient.
18. The pharmaceutical combination according to any one of items 1 to 9 for use in the treatment of cancer according to any one of items 12 to 17, wherein the HDM-2/p53 inhibitor, or a pharmaceutically acceptable salt thereof, is to be administered simultaneously or sequentially to an anaplastic lymphoma kinase (ALK) inhibitor, or a pharmaceutically acceptable salt thereof.
19. Use of a data carrier comprising information about using (i) a HDM-2/p53 inhibitor, or a pharmaceutically acceptable salt thereof, and (ii) an anaplastic lymphoma kinase (ALK) inhibitor, or a pharmaceutically acceptable salt thereof, simultaneously or sequentially, to instruct to administer (i) a HDM-2/p53 inhibitor, or a pharmaceutically acceptable salt thereof, and (ii) an anaplastic lymphoma kinase (ALK) inhibitor, or a pharmaceutically acceptable salt thereof, simultaneously or sequentially for the treatment of cancer.
20. A method of treating cancer in a patient comprising administering simultaneously or sequentially a therapeutically effective amount of (i) a HDM-2/p53 inhibitor, or a pharmaceutically acceptable salt thereof, and (ii) an anaplastic lymphoma kinase (ALK) inhibitor, or a pharmaceutically acceptable salt thereof.
21. The method of treating cancer in a patient according to item 20, wherein the cancer comprises mutated anaplastic lymphoma kinase (ALK).
22. The method of treating cancer in a patient according to item 20 or 21, wherein the cancer is neuroblastoma.
23. The method of treating cancer in a patient according to any one of items 20 to 22, wherein the cancer is relapsed or high-risk neuroblastoma.
24. The method of treating cancer in a patient according to any one of items 20 to 23, wherein the cancer comprises functional p53 or p53 wt.
25. The pharmaceutical combination according to any one of items 1 to 9 for the manufacture of a medicament or a pharmaceutical product for the treatment of cancer.
26. A HDM-2/p53 inhibitor, or a pharmaceutically acceptable salt thereof, and (ii) an anaplastic lymphoma kinase (ALK) inhibitor, or a pharmaceutically acceptable salt thereof, for combined use as a medicine.
27. The pharmaceutical combination according to any one of items 1 to 9, the pharmaceutical combination for use as a medicine according to items 10 or 11, the pharmaceutical combination for use in the treatment of cancer according to any one of items 12 to 18, the use of a data carrier according to item 19, the method of treating cancer in a patient according to any one of items 20 to 25, or the HDM-2/p53 inhibitor according to item 26, wherein the HDM-2/p53 inhibitor is a compound of formula (I) or formula (II) or a compound selected from a group consisting of:

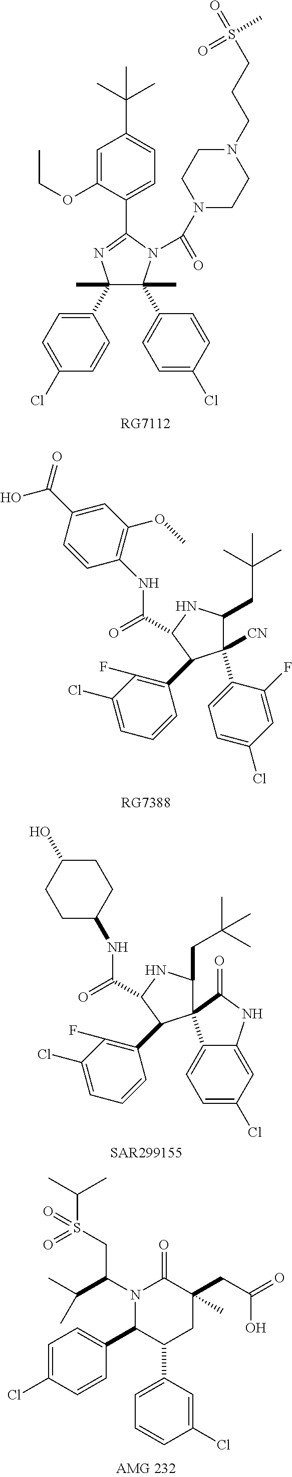

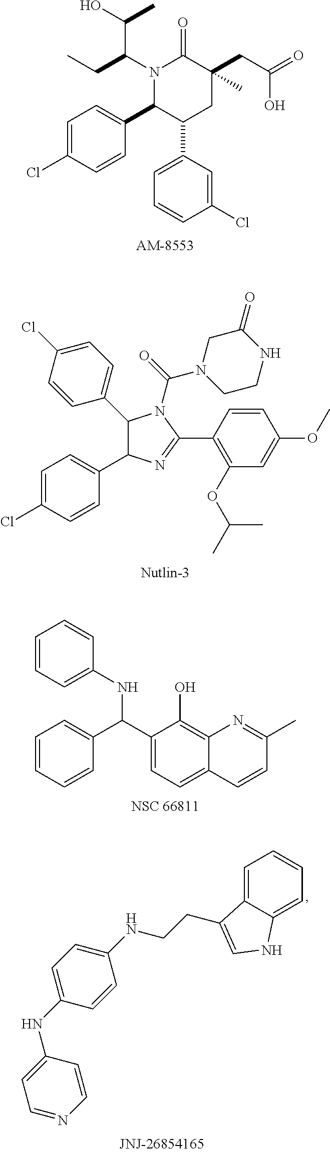

### Caylin-1, Caylin-2, HLI373, and SC204072.

28. The pharmaceutical combination according to any one of items 1 to 9, the pharmaceutical combination for use as a medicine according to items 10 or 11, the pharmaceutical combination for use in the treatment of cancer according to any one of items 12 to 18, the use of a data carrier according to item 19, the method of treating cancer in a patient according to any one of items 20 to 25, or the HDM-2/p53 inhibitor according to item 26, wherein the HDM-2/p53 inhibitor is selected from the group consisting of:
(S)-1-(4-Chloro-phenyl)-7-isopropoxy-6-methoxy-2-(4-{methyl-[4-(3-oxo-piperazin-1-yl)-trans-cyclohexylmethyl]-amino}-phenyl)-1,4-dihydro-2H-isoquinolin-3-one,
(S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one,
(S)-1-(4-Chloro-phenyl)-7-isopropoxy-6-methoxy-2-(6-{methyl-[4-(4-methyl-3-oxo-piperazin-1-yl)-trans-cyclohexylmethyl]-amino}-pyridin-3-yl)-1,4-dihydro-2H-isoquinolin-3-one,
(S)-1-(4-Chloro-phenyl)-7-isopropoxy-6-methoxy-2-(6-{methyl-[4-(3-methyl-4-oxo-imidazolidin-1-yl)-trans-cyclohexylmethyl]-amino}-pyridin-3-yl)-1,4-dihydro-2H-isoquinolin-3-one,
(S)-1-(4-Chloro-phenyl)-7-isopropoxy-6-methoxy-2-(5-{methyl-[4-(3-methyl-4-oxo-imidazolidin-1-yl)-trans-cyclohexylmethyl]-amino}-pyrazin-2-yl)-1,4-dihydro-2H-isoquinolin-3-one,
1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one,
(S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one,
4-[(S)-5-(3-Chloro-2-fluoro-phenyl)-2-(2,4-dimethoxy-pyrimidin-5-yl)-3-isopropyl-6-oxo-3,4,5,6-tetrahydro-pyrrolo[3,4-d]imidazol-4-yl]-benzonitrile,
(S)-5-(5-Chloro-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one,
(S)-5-(3-chloro-4-fluorophenyl)-6-(4-chlorophenyl)-2-(2,4-dimethoxy-pyrimidin-5-yl)-1-((R)-1-methoxypropan-2-yl)-5,6-dihydropyrrolo[3,4-d]imidazol-4(1H)-one, and
(S)-5-(5-chloro-1-methyl-2-oxo-1,2-dihydropyridin-3-yl)-6-(4-chlorophenyl)-2-(2,4-dimethoxy-d6-pyrimidin-5-yl)-1-((R)-1-methoxypropan-2-yl)-5,6-dihydropyrrolo[3,4-d]imidazol-4(1H)-one.
   29. The pharmaceutical combination according to any one of items 1 to 9, the pharmaceutical combination for use as a medicine according to items 10 or 11, the pharmaceutical combination for use in the treatment of cancer according to any one of items 12 to 18, the use of a data carrier according to item 19, the method of treating cancer in a patient according to any one of items 20 to 25, or the HDM-2/p53 inhibitor according to item 26, wherein the HDM-2/p53 inhibitor is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or pharmaceutically acceptable salt thereof.
   30. The pharmaceutical combination according to any one of items 1 to 9, the pharmaceutical combination for use as a medicine according to items 10 or 11, the pharmaceutical combination for use in the treatment of cancer according to any one of items 12 to 18, the use of a data carrier according to item 19, the method of treating cancer in a patient according to any one of items 20 to 25, or the HDM-2/p53 inhibitor according to item 26, wherein the HDM-2/p53 inhibitor is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, or pharmaceutically acceptable salt thereof.
   31. The pharmaceutical combination according to any one of items 1 to 9 or 27 to 30, the pharmaceutical combination for use as a medicine according to any one of items 10, 11 or 27 to 30, the pharmaceutical combination for use in the treatment of cancer according to any one of items 12 to 18 or 27 to 30, the use of a data carrier according to any one of items 19 or 27 to 30, the method of treating cancer in a patient according to any one of items 20 to 25 or 27 to 30, or the HDM-2/p53 inhibitor according to any one of items 26 or 27 to 30, wherein the anaplastic lymphoma kinase (ALK) inhibitor is selected from a group consisting of:

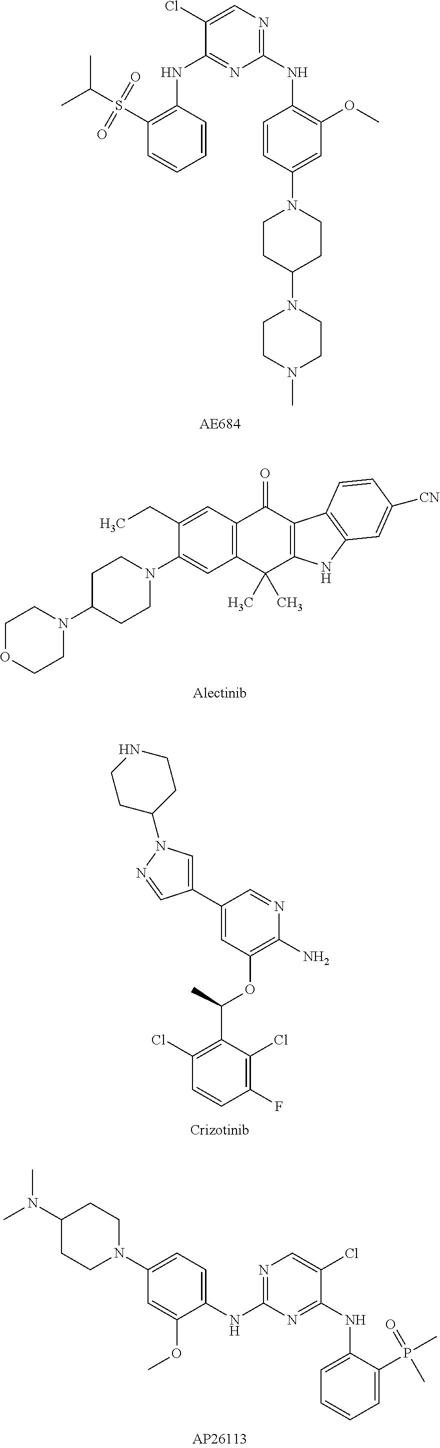

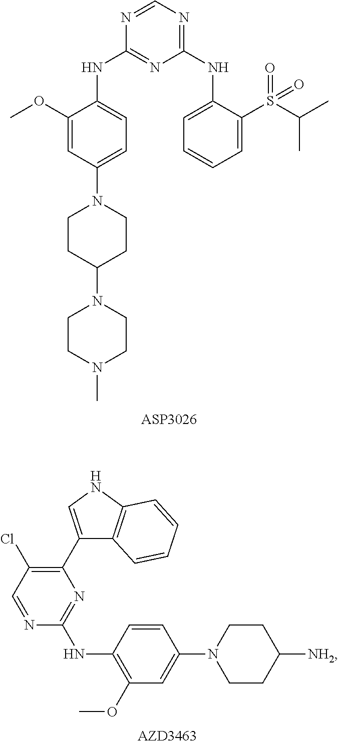 [SMILES: COc1cc(N2CCC(N3CCN(C)CC3)CC2)ccc1Nc1ncnc(Nc2ccccc2S(=O)(=O)C(C)C)n1 ; COc1cc(N2CCC(N)CC2)ccc1Nc1ncc(Cl)c(-c2c[nH]c3ccccc23)n1]

and 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof.
32. The pharmaceutical combination according to any one of items 1 to 9 or 27 to 30, the pharmaceutical combination for use as a medicine according to any one of items 10, 11 or 27 to 30, the pharmaceutical combination for use in the treatment of cancer according to any one of items 12 to 18 or 27 to 30, the use of a data carrier according to any one of items 19 or 27 to 30, the method of treating cancer in a patient according to any one of items 20 to 25 or 27 to 30, or the HDM-2/p53 inhibitor according to any one of items 26 or 27 to 30, wherein the anaplastic lymphoma kinase (ALK) inhibitor is 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof.
33. The pharmaceutical combination according to any one of items 1 to 9 or 27 to 32, the pharmaceutical combination for use as a medicine according to any one of items 10, 11 or 27 to 32, the pharmaceutical combination for use in the treatment of cancer according to any one of items 12 to 18 or 27 to 32, the use of a data carrier according to any one of items 19 or 27 to 32, the method of treating cancer in a patient according to any one of items 20 to 25 or 27 to 32, or the HDM-2/p53 inhibitor according to any one of items 26 or 27 to 32, further comprising another therapeutically active agent.
34. The pharmaceutical combination according to item 33, the pharmaceutical combination for use as a medicine according to item 33, the pharmaceutical combination for use in the treatment of cancer according to item 33, the use of a data carrier according to item 33, the method of treating cancer in a patient according to item 33, or the HDM-2/p53 inhibitor according to item 33, wherein the therapeutically active agent is an anti-cancer agent.
35. The pharmaceutical combination according to item 33 or 34, the pharmaceutical combination for use as a medicine according to item 33 or 34, the pharmaceutical combination for use in the treatment of cancer according to item 33 or 34, the use of a data carrier according to item 33 or 34, the method of treating cancer in a patient according to item 33 or 34, or the HDM-2/p53 inhibitor according to item 33 or 34, wherein the therapeutically active agent is a Cdk1-6 inhibitor, particularly Cdk 4/6 inhibitor, especially Cdk4 inhibitor.
36. The pharmaceutical combination according to any one of items 33 to 35, the pharmaceutical combination for use as a medicine according to any one of items 33 to 35, the pharmaceutical combination for use in the treatment of cancer according to any one of items 33 to 35, the use of a data carrier according to any one of items 33 to 35, the method of treating cancer in a patient according to any one of items 33 to 35, or the HDM-2/p53 inhibitor according to any one of items 33 to 35, wherein the therapeutically active agent is a compound selected from a group consisting of:

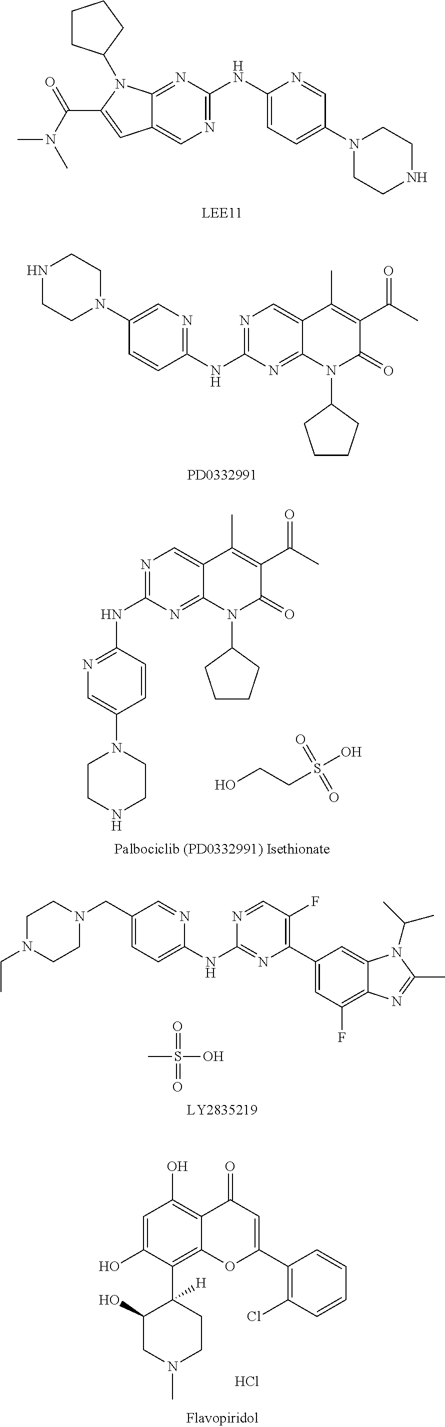

37. A pharmaceutical combination comprising or consisting of:
(a) an anaplastic lymphoma kinase (ALK) inhibitor, or a pharmaceutically acceptable salt thereof,
(b) at least one Braf inhibitor,
(c) or a pharmaceutically acceptable salt thereof.
   38. A pharmaceutical combination according to item 37, wherein the ALK inhibitor is 5-chloro-N2-(2-isopropoxy-5-methyl-4-(piperidin-4-yl)phenyl)-N4-[2-(propane-2-sulfonyl)-phenyl]-pyrimidine-2,4-diamine or a pharmaceutically acceptable salt thereof.
   39. A pharmaceutical combination according to item 37 or 38, wherein the BRAF inhibitor is selected from the group consisting of: (S)-methyl-1-(4-(3-(5-chloro-2-fluoro-3-(methylsulfonamido)phenyl)-1-isopropyl-1H-pyrazol-4-yl)pyrimidin-2-ylamino)propan-2-ylcarbamate;
methyl N-[(2S)-1-({4-[3-(5-chloro-2-fluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(2,5-difluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(5-chloro-2-fluoro-3-methanesulfonamidophenyl)-1-ethyl-1H-pyrazol-4-yl]pyrimidin-2-yl}amino)propan-2-yl]carbamate;
methyl N-[(2S)-1-({4-[3-(2-fluoro-3-methanesulfonamido-5-methylphenyl)-1-(propan-2-yl)-1H-pyrazol-4-yl]pyrimidin-2-yl}amino)propan-2-yl]carbamate;
methyl N-[(2S)-1-({4-[3-(2-chloro-3-methanesulfonamido-5-methylphenyl)-1-(propan-2-yl)-1H-pyrazol-4-yl]pyrimidin-2-yl}amino)propan-2-yl]carbamate;
methyl N-[(2S)-1-({4-[3-(2-chloro-5-fluoro-3-methanesulfonamidophenyl)-1-(propan-2-yl)-1H-pyrazol-4-yl]pyrimidin-2-yl}amino)propan-2-yl]carbamate;
methyl N-[(2R)-1-({4-[3-(5-chloro-2-fluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(2,5-dichloro-3-methanesulfonamidophenyl)-1-(propan-2-yl)-1H-pyrazol-4-yl]pyrimidin-2-yl}amino)propan-2-yl]carbamate; and
vemurafenib.
   40. A pharmaceutical combination according to any one of items 37 to 39, wherein the BRAF inhibitor is S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate.
   41. A pharmaceutical combination according to any one of items 37 to 40 in the form of a pharmaceutical product or pharmaceutical composition.
   42. A pharmaceutical combination according to any one of items 37 to 41 for use in the treatment of melanoma, lung cancer or neuroblastoma.

In one embodiment, the present disclosure relates to a combined preparation which comprises: (i) one or more unit dosage forms of combination partner (a), and (ii) one or more unit dosage forms of combination partner (b). The present disclosure particularly pertains to a pharmaceutical combination comprising (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier useful for treating or preventing a proliferative disease in a subject in need thereof.

The present disclosure also pertains to a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition or medicament for the treatment or prevention of a proliferative disease in a subject in need thereof.

The present disclosure also pertains to a pharmaceutical combination comprising: (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof, and optionally at least one pharmaceutically acceptable carrier useful for treating or preventing a proliferative disease in a subject in need thereof.

The present disclosure further pertains to the use of (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof for the preparation of a pharmaceutical composition or medicament for the treatment or prevention of a proliferative disease.

The present disclosure relates to a method of treating a subject having a proliferative disease, namely cancer, comprising the step of administering to said subject a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier in a quantity, which is jointly therapeutically effective against the proliferative disease or cancer.

The present disclosure further provides a commercial package comprising as therapeutic agents a combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition, together with instructions for simultaneous, separate or sequential administration thereof for use in the delay of progression or treatment of a proliferative disease.

The above combinations are also provided for simultaneous, separate or sequential administration, in particular for treating or preventing a proliferative disease.

### BRIEF DESCRIPTION OF THE FIGURES

FIG. 1 summarizes the genetic risks associated with high risk neuroblastomas.

FIG. 2 summarizes and compares the respective sensitivities of LDK378 and crizotinib as monotherapy in neuroblastomas ALK+ neuroblastoma cell lines (cell line NB-1 in FIG. 2(a) and cell line SH-SY5Y in FIG. 2(b)).

FIG. 3 summarizes that LDK378 (ALK inhibitor) and HDM-2/P53 Inhibitor Combination Promotes Apoptosis in ALK+ and TP53 WT NB Cell Lines.

FIGS. 4(a) and 4(b) summarizes that HDM-2 (or related MDM-2) inhibitors down-regulate MYCN in MYCN-amplified neuroblastoma cell lines.

FIG. 5 summarizes data for treatments with LDK378 (ALK inhibitor) and CGM097 (HDM-2/P53 Inhibitor) as single agents and in combination in a NB-1 Xenograft.

FIG. 6 summarizes data for treatments with LDK378 (ALK inhibitor) and CGM097 (HDM-2/P53 Inhibitor) as single agents and in combination in a SY5Y Xenograft.

FIG. 7 summarizes data for LDK378 Single Agent and Combination Treatments with NVP-CGM097 in a NB-1 Xenograft. It also shows the use of treatment of LDK378 (ALK inhibitor) and CGM097 (HDM-2/P53 Inhibitor) in combination with a further therapeutic co-agent. Under continuous treatment, tumors in LDK378 single agent treated group and LDK378+LEE011 treated group resumed growth before day 41. Tumors in LDK378+CGM097 treated group and LDK378+CGM097+LEE011 treated group remained small under treatment. After treatment termination, tumors resumed growth.

FIG. 8 summarizes efficacy (A) and safety (B) data for LDK378 and Compound C as single agents, as well as for the combination treatment with LDK378 and Compound C in NB-1 neuroblastoma xenograft model.

### DETAILED DESCRIPTION OF THE DISCLOSURE

The present disclosure provides a pharmaceutical combination comprising (i) a HDM-2/p53 inhibitor, or a pharmaceutically acceptable salt thereof, and (ii) an anaplastic lymphoma kinase (ALK) inhibitor, or a pharmaceutically acceptable salt thereof.

More specifically, the present disclosure provides pharmaceutical combinations comprising: (a) an anaplastic lymphoma kinase (ALK) inhibitor, namely 5-chloro-N2-(2-isopropoxy-5-methyl-4-(piperidin-4-yl)phenyl)-N4-[2-(propane-2-sulfonyl)-phenyl]-pyrimidine-2,4-diamine, and (b) at least one HDM2/p53 (or related Mdm-2) inhibitor selected from the group comprising NVP-CGM097; Caylin-1, Caylin-2, HLI373, Nutlin-3; SC204072 or a pharmaceutically acceptable salt thereof; or at least one BRaf inhibitor selected from LGX818, and optionally at least one pharmaceutically acceptable carrier.

In one embodiment, the present disclosure relates to a combined preparation which comprises: (i) one or more unit dosage forms of combination partner (a), and (ii) one or more unit dosage forms of combination partner (b). The present disclosure particularly pertains to a pharmaceutical combination comprising (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier useful for treating or preventing a proliferative disease in a subject in need thereof.

The present disclosure also pertains to a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition or medicament for the treatment or prevention of a proliferative disease in a subject in need thereof.

The present disclosure also pertains to a pharmaceutical combination comprising: (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof, and optionally at least one pharmaceutically acceptable carrier useful for treating or preventing a proliferative disease in a subject in need thereof.

The present disclosure further pertains to the use of (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof for the preparation of a pharmaceutical composition or medicament for the treatment or prevention of a proliferative disease.

The present disclosure relates to a method of treating a subject having a proliferative disease, namely cancer, comprising the step of administering to said subject a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier in a quantity, which is jointly therapeutically effective against the proliferative disease or cancer.

The present disclosure further provides a commercial package comprising as therapeutic agents a combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition, together with instructions for simultaneous, separate or sequential administration thereof for use in the delay of progression or treatment of a proliferative disease.

The above combinations are also provided for simultaneous, separate or sequential administration, in particular for treating or preventing a proliferative disease.

The present disclosure relates to such pharmaceutical combinations for simultaneous, separate or sequential administration, in particular for use in the treatment or prevention of a proliferative disease, namely cancer.

The general terms used herein are defined with the following meanings, unless explicitly stated otherwise:

The terms “comprising” and “including” are used herein in their open-ended and non-limiting sense unless otherwise noted.

The present disclosure embodiments also include pharmaceutically acceptable salts of the compounds useful according to the disclosure described herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. Suitable organic acids are, e.g., carboxylic acids or sulfonic acids, such as acetic acid, succinic acid, fumaric acid or methansulfonic acid. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. For example, the salt is a sulphate salt, or bisulphate salt. In another embodiment, the salt is a succinic salt.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds useful according to the disclosure (=being included in a combination, especially a pharmaceutical combination, according to the disclosure, respectively, or being used according to the disclosure, optionally also including further co-agents as defined below, that is, all active ingredients), as well as their pharmaceutically acceptable salts, can also be present as tautomers, N-oxides or solvates, e.g. hydrates. All these variants, as well as any single one thereof or combination of two or more to less than all such variants, are encompassed and to be read herein where a compound included in the inventive combination products, e.g. a HDM-2/p53 inhibitor, an anaplastic lymphoma kinase (ALK) inhibitor and/or a BRAF inhibitor, is mentioned.

The present disclosure, according to a first embodiment mentioned above and below, relates to a pharmaceutical combination, especially a pharmaceutical combination product, comprising the mentioned combination partners and at least one pharmaceutically acceptable carrier.

“Pharmaceutical combination” refers to use, application or formulations of the separate partners with or without instructions for combined use or to combination products. The combination partners may thus administered entirely separately or be entirely separate pharmaceutical dosage forms. The combination partners may be pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active, especially as defined below. It can refer to either a fixed combination in one dosage unit form, or a kit of parts for the combined administration where an HDM-2/p53 inhibitor and an anaplastic lymphoma kinase (ALK) inhibitor, or an anaplastic lymphoma kinase (ALK) inhibitor and a BRAF inhibitor (and optionally yet a further combination partner (e.g. another drug as explained below, also referred to as “co-agent”) may be administered independently at the same time or separately within time intervals, especially where these time intervals allow that the combination partners show a cooperative (=joint) effect. In one embodiment the effect is synergistic.

The terms “co-administration” or “combined administration” or “combined use” or the like as utilized herein are meant to encompass administration of the selected combination partner to a single subject in need thereof (e.g. a patient), and are intended to include treatment regimens in which the agents are not necessarily administered by the same route of administration and/or at the same time.

The term “fixed combination” means that the active ingredients, e.g. an HDM-2/p53 inhibitor and an anaplastic lymphoma kinase (ALK) inhibitor (or an anaplastic lymphoma kinase (ALK) inhibitor and a BRAF inhibitor) are both administered to a patient simultaneously in the form of a single entity or dosage. In other terms: the active ingredients are present in one dosage form, e.g. in one tablet or in one capsule.

The term “non-fixed combination” means that the active ingredients are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients. The term “non-fixed combination” thus defines especially administration, use, composition or formulation in the sense that the combination partners, for example (i) HDM-2/p53 inhibitor and (ii) an anaplastic lymphoma kinase (ALK) inhibitor (and if present further one or more co-agents) as defined herein can be dosed independently of each other or by use of different fixed combinations with distinguished amounts of the combination partners, i.e. simultaneously or at different time points, where the combination partners may also be used as entirely separate pharmaceutical dosage forms or pharmaceutical formulations that are also sold independently of each other and just instructions of the possibility of their combined use is or are provided in the package equipment, e.g. leaflet or the like, or in other information e.g. provided to physicians and medical staff. The independent formulations or the parts of the formulation, product, or composition, can then, e.g. be administered simultaneously or chronologically staggered, that is at different time points and with equal or different time intervals for any part of the kit of parts. Particularly, the time intervals are chosen such that the effect on the treated disease in the combined use of the parts is larger than the effect which would be obtained by use of only any one of the combination partners (i) and (ii), thus being jointly active. The ratio of the total amounts of the combination partner (i) to the combination partner (ii) to be administered in the combined preparation can be varied, e.g. in order to cope with the needs of a patient sub-population to be treated or the needs of the single patient which different needs can be due to age, sex, body weight, etc. of the patients.

The terms “a” and “an” and “the” and similar references in the context of describing the disclosure (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “pharmaceutical composition” is defined herein to refer to a mixture or solution containing at least one therapeutic agent to be administered to a subject, e.g., a mammal or human, in order to prevent or treat a particular disease or condition affecting the mammal or human.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject, e.g., a mammal or human. The term “prevent”, “preventing” or “prevention” as used herein comprises the prevention of at least one symptom associated with or caused by the state, disease or disorder being prevented.

The term “jointly therapeutically active” or “joint therapeutic effect” as used herein means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both compounds are present in the blood of the human to be treated at least during certain time intervals.

The term “pharmaceutically effective amount” or “clinically effective amount” of a combination of therapeutic agents is an amount sufficient to provide an observable improvement over the baseline clinically observable signs and symptoms of the disorder treated with the combination.

The term “synergistic effect” as used herein refers to action of two therapeutic agents such as, for example, a compound CGM097 as the HDMA-2/p53 inhibitor and LDK378 as the an anaplastic lymphoma kinase (ALK) inhibitor, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from cancers.

The term about” or “approximately” shall have the meaning of within 10%, more preferably within 5%, of a given value or range.

The following definitions show more specific embodiments of general features or expressions which can be used to replace one, more than one or all general features or expressions in the disclosure embodiments described hereinbefore and hereinafter, thus leading to more specific disclosure embodiments.

According to the present disclosure the HDM-2/p53 inhibitor can be any compound inhibiting the HDM-2/p53 interaction with an IC₅₀ of less than 100 μM, preferably less than 10 μM, measured by a Time Resolved Fluorescence Energy Transfer (TR-FRET) Assay. The inhibition of p53-Hdm2 and p53-Hdm4 interactions is measured by time resolved fluorescence energy transfer (TR-FRET). Fluorescence energy transfer (or Foerster resonance energy transfer) describes an energy transfer between donor and acceptor 5 fluorescent molecules. For this assay, MDM2 protein (amino acids 2-188) and MDM4 protein (amino acids 2-185), tagged with a C-terminal Biotin moiety, are used in combination with a Europium labeled streptavidin (Perkin Elmer, Inc., Waltham, Mass., USA) serving as the donor fluorophore. The p53 derived, Cy5 labeled peptide Cy5-TFSDLWKLL (p53 aa18-26) is the energy acceptor. Upon excitation of the donor 10 molecule at 340 nm, binding interaction between MDM2 or MDM4 and the p53 peptide induces energy transfer and enhanced response at the acceptor emission wavelength at 665 nm. Disruption of the formation of the p53-MDM2 or p53-MDM4 complex due to an inhibitor molecule binding to the p53 binding site of MDM2 or MDM4 results in increased donor emission at 615 nm. The ratiometric FRET assay readout is calculated from the 15 raw data of the two distinct fluorescence signals measured in time resolved mode (countrate 665 nm/countrate 615 nm×1000). The assay can be performed according to the following procedure: The test is performed in white 1536w microtiterplates (Greiner Bio-One GmbH, Frickenhausen, Germany) in a total volume of 3.1 μl by combining 100 μl of compounds diluted in 90% DMSO/10% H2O (3.2% final DMSO concentration) with 2 μl Europium 20 labeled streptavidin (final concentration 2.5 nM) in reaction buffer (PBS, 125 mM NaCl, 0.001% Novexin (consists of carbohydrate polymers (Novexin polymers), designed to increase the solubility and stability of proteins; Novexin Ltd., ambridgeshire, United Kingdom), Gelatin 0.01%, 0.2% Pluronic (block copolymer from ethylenoxide and propyleneoxide, BASF, Ludwigshafen, Germany), 1 mM DTT), followed by the addition of 0.5 μl MDM2-Bio or MDM4-Bio diluted in assay buffer (final concentration 10 nM). Allow the solution to pre-incubate for 15 minutes at room temperature, followed by addition of 0.5 μl Cy5-p53 peptide in assay buffer (final concentration 20 nM). Incubate at room temperature for 10 minutes prior to reading the plate. For measurement of samples, an Analyst GT multimode microplate reader (Molecular Devices) with the following settings 30 is used: Dichroic mirror 380 nm, Excitation 330 nm, Emission Donor 615 nm and Emission Acceptor 665 nm. IC50 values are calculated by curve fitting using XLfit. If not specified, reagents are purchased from Sigma Chemical Co, St. Louis, Mo., USA.

An HDM-2/p53 inhibitor can be a compound of formula (I):

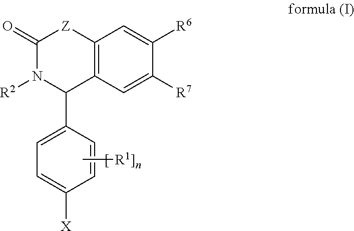

wherein
Z is CH₂ or N—R⁴;
X is halogen;
R⁴ is selected from the group consisting of

### H—

C₁-C₇-alkyl-;
R⁶ is independently selected from the group consisting of

### H—

### R′O—

### (R′)₂N—;

R⁷ is independently selected from the group consisting of

### R′O—

### (R′)₂N—;

each R′ is independently selected from the group consisting of

### H—

C₁-C₇alkenyl-
halo-C₁-C₇alkyl-
halo-C₁-C₇alkenyl-
C₃-C₁₂-cycloalkyl-
heterocyclyl-
aryl-
hydroxy-C₁-C₇alkyl-
C₁-C₇alkoxy-C₁-C₇alkyl-
amino-C₁-C₇alkyl-
N—C₁-C₇alkyl-amino-C₁-C₇alkyl-
N,N-di-C₁-C₇alkyl-amino-C₁-C₇alkyl-
C₃-C₁₂-cycloalkyl-C₁-C₇alkyl-
heterocyclyl-C₁-C₇alkyl-
aryl-C₁-C₇alkyl-
C₁-C₇alkyl-carbonyl-
halo-C₁-C₇alkyl-carbonyl-
hydroxy-C₁-C₇alkyl-carbonyl-
C₁-C₇alkoxy-C₁-C₇alkyl-carbonyl-
amino-C₁-C₇alkyl-carbonyl-
N—C₁-C₇alkyl-amino-C₁-C₇alkyl-carbonyl-
N,N-di-C₁-C₇alkyl-amino-C₁-C₇alkyl-carbonyl-
C₃-C₁₂-cycloalkyl-carbonyl-
heterocyclyl-C₁-C₇alkyl-carbonyl-
aryl-C₁-C₇alkyl-carbonyl-
C₃-C₁₂-cycloalkyl-C₁-C₇alkyl-carbonyl-
heterocyclyl-carbonyl-
aryl-carbonyl-
C₁-C₇alkyl-carbonyl-C₁-C₇alkyl-
halo-C₁-C₇alkyl-carbonyl-C₁-C₇alkyl-
hydroxy-C₁-C₇alkyl-carbonyl-C₁-C₇alkyl-
C₁-C₇alkoxy-C₁-C₇alkyl-carbonyl-C₁-C₇alkyl-
amino-C₁-C₇alkyl-carbonyl-C₁-C₇alkyl-
N—C₁-C₇alkyl-amino-C₁-C₇alkyl-carbonyl-C₁-C₇alkyl-
N,N-di-C₁-C₇alkyl-amino-C₁-C₇alkyl-carbonyl-C₁-C₇alkyl-
C₃-C₁₂-cycloalkyl-carbonyl-C₁-C₇alkyl-
heterocyclyl-carbonyl-C₁-C₇-alkyl-
aryl-carbonyl-C₁-C₇alkyl-
carbonyl-C₁-C₇alkyl-
hydroxy-carbonyl-C₁-C₇alkyl-
C₁-C₇alkoxy-carbonyl-C₁-C₇alkyl-
amino-carbonyl-C₁-C₇alkyl-
N—C₁-C₇alkyl-amino-carbonyl-C₁-C₇alkyl-
N,N-di-C₁-C₇alkyl-amino-carbonyl-C₁-C₇alkyl-
C₃-C₁₂-cycloalkyl-carbonyl-C₁-C₇alkyl-
heterocyclyl-carbonyl-C₁-C₇-alkyl-
aryl-carbonyl-C₁-C₇alkyl-
C₁-C₇alkyl-carbonyl-amino-C₁-C₇alkyl-
C₁-C₇alkyl-carbonyl-N—C₁-C₇alkyl-amino-C₁-C₇alkyl-
halo-C₁-C₇alkyl-carbonyl-amino-C₁-C₇alkyl-
halo-C₁-C₇alkyl-carbonyl-N—C₁-C₇alkyl-amino-C₁-C₇alkyl-
wherein aryl, heterocyclyl and C₃-C₁₂-cycloalkyl are unsubstituted or substituted by 1-4 substituents selected from C₃-C₇-alkyl, halo-C₁-C₇alkyl, halogen, hydroxy, C₁-C₇alkoxy,
amino, nitro or cyano;
   each R¹ is independently selected from the group consisting of
   halogen-
   cyano-
   nitro-
   C₁-C₇alkenyl-
   halo-C₁-C₇alkyl-
   hydroxy-
   C₁-C₇alkoxy-
   amino-
   N—C₁-C₇alkyl-amino-
   N,N-di-C₁-C₇alkyl-amino-
   amino-carbonyl-amino-
   N—C₁-C₇alkyl-amino-carbonyl-amino-
   N,N-di-C₁-C₇alkyl-amino-carbonyl-amino-
   C₁-C₇alkyl-carbonyl-amino-
   amino-carbonyl-
   N—C₁-C₇alkyl-amino-carbonyl-
   N,N-di-C₁-C₇alkyl-amino-carbonyl-
   hydroxy-C₁-C₇alkyl-
   amino-C₁-C₇alkyl-
   N—C₁-C₇alkyl-amino-C₁-C₇alkyl-
   N,N-di-C₁-C₇alkyl-amino-C₁-C₇alkyl-
   C₁-C₇alkyl-carbonyl-amino-C₁-C₇alkyl-
   C₁-C₇alkyl-carbonyl-N—C₁-C₇alkyl-amino-C₁-C₇alkyl-;
   n is 0 to 2;
   R² is selected from
(A) phenyl, 2-pyridyl and 3-pyridyl
   substituted in the para-position relative to the isoquinolinone or quinazolinone, by (R³)₂N—Y—
      wherein Y is absent (a bond) or
   (R³)₂N—Y— is selected from

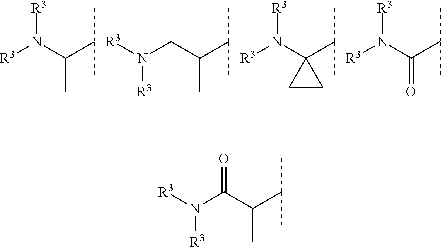

and wherein said phenyl, 2-pyridyl or 3-pyridyl is optionally substituted by 1-2 additional substituents selected from
halogen-
cyano-
halo-C₁-C₇alkyl-
hydroxy-
C₁-C₇-alkoxy- and
hydroxy-C₁-C₇-alkyl-;
   or
(B) phenyl, 2-pyridyl or 3-pyridyl
   substituted in para-position relative to the isoquinolinone or quinazolinone by a substituent selected from
   cyano-
   halogen-
   nitro-
   halo-C₁-C₇alkyl-
   hydroxy-C₁-C₇alkyl-
   hydroxy-carbonyl-
   C₁-C₇alkoxy-carbonyl-
   C₁-C₇alkyl-carbonyl-
   C₁-C₇alkoxy-
   (C-bound)-heterocyclyl-
      wherein (C-bound)-heterocyclyl is unsubstituted or substituted by 1-4 substituents selected from C₁-C₇-alkyl, halo-C₁-C₇alkyl, halogen, hydroxy, C₁-C₇alkoxy, amino, nitro or cyano;
   and optionally substituted by 1-2 additional substituents selected from
   halogen-
   cyano-
   halo-C₁-C₇alkyl-
   hydroxy-
   C₁-C₇alkoxy-
   (C-bound or N-bound)heterocyclyl-C₁-C₄-alkyl-
   hydroxy-C₁-C₇-alkyl-;
      or
(C) phenyl,
   substituted in ortho-position relative to the isoquinolinone or quinazolinone by R³O—
      and substituted in para- or meta-position by a substituent selected from methyl, chloro, C₁-C₇-alkyl-carbonyl- or C₁-C₇-alkoxy-carbonyl-;
(D) (C-bound)-heterocycle selected from

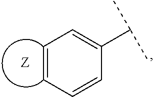

wherein Z is a 4-6 membered heterocyclic ring, annulated to phenyl in para and meta position, containing 1-3 heteroatoms selected from N, O or S,
   which is optionally substituted by 1-2 additional substituents selected from
   halogen-
   cyano-
   C₁-C₇alkyl-
   halo-C₁-C₇alkyl-
   hydroxy-
   C₁-C₇alkoxy-
   hydroxy-C₁-C₇alkyl-;
(E) pyrazin-2-yl,
substituted at the 5 position by:

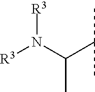

(F) pyridazin-3-yl, substituted at the 6 position by:

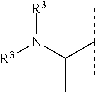

or
(G) pyrimidin-2-yl, substituted at the 5 position by:

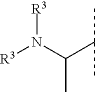

wherein each R³ is independently selected from
H—
C₁-C₇-alkyl-
hydroxy-C₁-C₇alkyl-
C₃-C₁₂-cycloalkyl-
C₁-C₇alkoxy-C₁-C₇alkyl-carbonyl-
amino-C₁-C₇alkyl-carbonyl
N—C₁-C₇-alkyl-amino-C₁-C₇alkyl-carbonyl
N,N-di C₁-C₇alkyl-amino-C₁-C₇alkyl-carbonyl
(R⁵)₂N—C₃-C₁₂-cycloalkyl-
(R⁵)₂N—C₁-C₇alkyl-
(R⁵)₂N—C₃-C₁₂-cycloalkyl-C₁-C₇alkyl-
(R⁵)₂N—C₃-C₁₂-cycloalkyl-carbonyl-
R⁵O—C₃-C₁₂-cycloalkyl-
R⁵O—C₁-C₇alkyl-
R⁵O—C₃-C₁₂-cycloalkyl-C₁-C₇alkyl-
R⁵O—(C₁-C₇alkyl)-C₃-C₁₂-cycloalkyl-C₁-C₇alkyl-
R⁵O-(hydroxy-C₁-C₇alkyl)-C₃-C₁₂-cycloalkyl-C₁-C₇alkyl-
(R⁵)₂N—CO—C₃-C₁₂-cycloalkyl-C₁-C₇alkyl-
C₁-C₇alkoxycarbonyl-C₃-C₁₂-cycloalkyl-C₁-C₇alkyl-
hydroxycarbonyl-C₃-C₁₂-cycloalkyl-C₁-C₇alkyl-
amino-carbonyl-C₃-C₁₂-cycloalkyl-C₁-C₇alkyl-
R⁵O—C₃-C₁₂-cycloalkyl-carbonyl-
(R⁵)₂N-carbonyl-C₁-C₇alkyl-
R⁵O-carbonyl-C₁-C₇alkyl-
aryl-C₁-C₇-alkyl-
heterocyclyl-C₁-C₇alkyl-
C₁-C₇-alkyl-carbonyl-
halo-C₁-C₇alkyl-carbonyl-
heterocyclyl-carbonyl-
aryl-carbonyl-
C₃-C₁₂-cycloalkyl-carbonyl-
C₃-C₁₂-cycloalkyl-C₁-C₇alkyl-
heterocyclyl-
aryl-
   wherein aryl, heterocyclyl and C₃-C₁₂-cycloalkyl are unsubstituted or substituted by 1-4 substituents selected from
   halogen-
   C₁-C₇alkyl-
   halo-C₁-C₇alkyl-
   C₁-C₇alkyl-carbonyl-
   C₃-C₁₂-cycloalkyl-carbonyl-
   C₁-C₇alkyl-sulfonyl-
   amino-sulfonyl-
   N—C₁-C₇alkyl-amino-sulfonyl-
   N,N-di-C₁-C₇alkyl-amino-sulfonyl-
   amino-carbonyl-
   N—C₁-C₇alkyl-amino-carbonyl-
   N,N-di-C₁-C₇alkyl-amino-carbonyl-
   oxo=
or
two R³, together with the N to which they are attached my form a 3-9 membered heterocyclic ring, optionally containing 1-4 additional heteroatoms selected from N, O or S, said heterocyclic ring is unsubstituted or substituted by 1-3 substituents selected from:
halogen-
hydroxy-
halo-C₁-C₇alkyl-
oxo=
hydroxy-
C₁-C₇alkoxy-
amino-
N—C₁-C₇alkyl-amino-
hydroxy-carbonyl-
C₁-C₇alkoxy-carbonyl-
amino-carbonyl-
N—C₁-C₇alkyl-amino-carbonyl-
N,N-di-C₁-C₇alkyl-amino-carbonyl-
C₁-C₇alkyl-carbonyl-
C₁-C₇alkyl-sulphonyl-
heterocyclyl-
C₁-C₇alkyl-carbonyl-amino-
C₁-C₇alkyl-carbonyl-N—C₁-C₇alkyl-amino-;
and
   each R⁵ is independently selected from:
H—
hydroxy-C₁-C₇alkyl-
C₁-C₇alkyl-carbonyl-
C₁-C₇alkoxy-carbonyl-C₁-C₇alkyl-
amino-carbonyl-C₁-C₇alkyl-
N—C₁-C₇alkyl-amino-carbonyl-C₁-C₇alkyl-
N,N-di-C₁-C₇alkyl-amino-carbonyl-C₁-C₇alkyl-
C₁-C₇alkyl-sulfonyl-
amino-sulfonyl-
N—C₁-C₇alkyl-amino-sulfonyl-
N,N-di-C₁-C₇alkyl-amino-sulfonyl-
heterocyclyl-carbonyl-
amino-carbonyl-
N—C₁-C₇alkyl-amino-carbonyl-
N,N-di-C₁-C₇alkyl-amino-carbonyl-
C₃-C₁₂-cycloalkyl-carbonyl-
C₁-C₇alkoxy-carbonyl-amino-C₁-C₇alkyl-
C₁-C₇alkoxy-carbonyl-N—C₁-C₇alkyl-amino-C₁-C₇alkyl-
C₁-C₇alkoxy-carbonyl-
C₃-C₁₂-cycloalkyl-
hydroxy-C₃-C₁₂-cycloalkyl-
or
two R⁵, together with the N to which they are attached my form a 3-9 membered heterocyclic ring, optionally containing from 1-4 additional heteroatoms selected from N, O or S, said heterocyclic ring is unsubstituted or substituted by from 1 to 3 substituents selected from
C₁-C₇alkyl-
oxo=,
C₁-C₇alkyl-carbonyl,
C₁-C₇-alkyl-sulphonyl,
hydroxy-C₁-C₇alkyl;
   with the proviso that if Z is CH₂, n is 0 or 1, and when present, R¹ is ortho-chloro, and R² is selected from
para-C₁-C₃-alkyl-phenyl-
para-(halo-C₁-C₃-alkyl)-phenyl-
para-C₁-C₃-alkoxy-phenyl-
para-halo-phenyl-
para-nitro-phenyl-
para-(C₁-C₃-alkoxy-carbonyl)-phenyl-
para-(hydroxy-carbonyl)-phenyl-
wherein the phenyl is optionally substituted by 1-2 additional substituents, said
substituents being independently selected from halo and methyl,
   then R⁶ and R⁷ are not both ethoxy or methoxy,
   aryl means phenyl or naphthyl,
   and
   heterocyclyl means an unsaturated, saturated, or partially saturated ring or ring system comprising 3, 4, 5, 6, 7, 8, 9, 10, 11 or 12 ring atoms, and containing at least one heteroatom selected from N, O and S, where the N and S can also optionally be oxidized, and wherein, unless otherwise stated, the heterocyclic group can be attached at a heteroatom or a carbon atom. The compounds can be synthetized as explained in WO 2011/076786. The reference also includes specific examples of possible compounds.

The HDM-2/p53 inhibitor can also be a compound of formula (II):

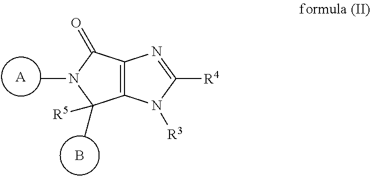

wherein

A is selected from:

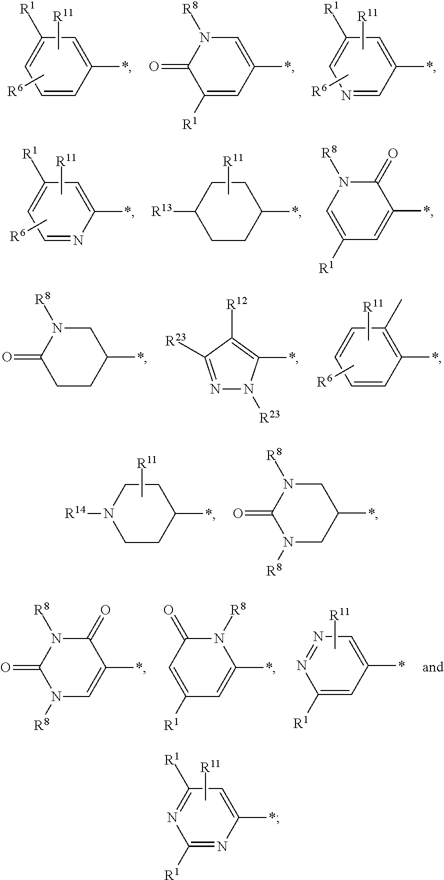

B is selected from:

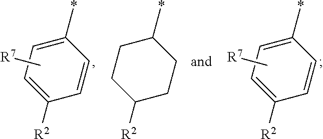

each R¹ is independently selected from halo and methyl;
R² is selected from chloro, fluoro, trifluoromethyl, methyl and cyano;
R³ is selected from isopropyl, cyclopropyl, isobutyl, cyclobutyl and cyclopentyl, or R³ is:

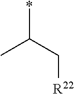

wherein R²² is selected from OH, OCH₃, NH₂, NHMe, NMe₂, NHCOMe and NHCOH;
R⁴ is selected from:

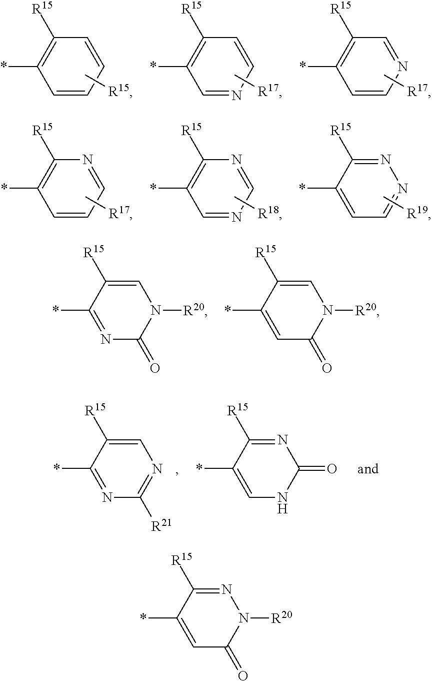

wherein
R¹⁵ is independently selected from OCH₃, CH₂CH₃, OH, OCF₃ and H;
R¹⁶ is selected from H, —O—(C₁-C₄)alkyl, halo, OCF₃, CN, —C(O)NR⁹R¹⁰, —C(O)— morpholinyl-4-yl, hydroxy-azetidin-1-yl-carbonyl, —CH₂NR⁹R¹⁰, —CH₂NR⁹—C(O)R¹⁰, CH₂CN, methyl-imidazolyl-, —CH₂C(O)NR⁹R¹⁰, —CH₂C(O)OH, —C(O)OH, —CH₂C(O)O—(C₁-C₄)alkyl, —N(R⁹)—C(O)—(C₁-C₄)alkyl, —NR⁹R¹⁰ and (C₁-C₄)alkyl optionally substituted by 1 or 2 OH;
R¹⁷ is selected from H, O(C₁-C₄)alkyl, —CH₂C(O)NR⁹R¹⁰, —CH₂C(O)O—(C₁-C₄)alkyl, —CH₂C(O)OH, —NR⁹R¹⁰, —C(O)NR⁹R¹⁰, —CH₂NR⁹R¹⁰, —C(O)OCH₃ and —CH₂CN;
R¹⁸ is selected from H, O(C₁-C₄)alkyl, OH, CH₂NR⁹R¹⁰, —NR⁹R¹⁰ and azetidin-1-yl, said azetidin-1-yl being substituted with OH or both CH₃ and OH,
R¹⁹ is selected from H, O(C₁-C₄)alkyl, (C₁-C₄)alkyl, —NR⁹R¹⁰, —N(R⁹)—C(O)—(C₁-C₄)alkyl and —C(O)NR⁹R¹⁰;
R²⁰ is selected from H, CH₃ and —CH₂CH₃;
R²¹ is selected from —NR⁹R¹⁰, —CH₂NR⁹R¹⁰, C(O)NR⁹R¹⁰ and CN;
R⁵ is selected from:
H,
heterocyclyl¹-C(O)—(CH₂)ₙ—,
(C₁-C₄)alkyl-, said (C₁-C₄)alkyl- being optionally substituted with 1 or 2 substituents independently selected from OH, ═O,
heterocyclyl¹-(C₁-C₄)alkyl-, wherein said alkyl of heterocyclyl¹-(C₁-C₄)alkyl- is optionally substituted by 1 or 2 OH, and said heterocyclyl¹ can be optionally substituted by methyl or ethyl,
(C₁-C₄)alkyl-O—C(O)—(CH₂)ₘ—, and
cyano;
   R⁶ is selected from:
H,
(C₁-C₄)alkyl-, optionally substituted with (C₁-C₄)alkoxy,
(C₁-C₄)alkoxy, optionally substituted with (C₁-C₄)alkoxy,
(C₁-C₄)alkoxy(C₁-C₄)alkoxy(C₁-C₄)alkyl-,
halo,
R⁹(R¹⁰)N—C(O)—(CH₂)ₘ—,
cyano,
R⁹(R¹⁰)N—(CH₂)ₘ—,
R⁹(R¹⁰)N—(CH₂)ₙ—O—(CH₂)ₘ—,
(C₁-C₄)alkyl-C(O)—(R¹⁰)N—(CH₂)ₘ—,
—O—(CH₂)ₚ-heteroaryl²;
   R⁷ is selected from:
H,
halo, and
(C₁-C₄)alkyl-, optionally substituted with (C₁-C₄)alkoxy;
   each R⁸ is independently selected from H, methyl, ethyl, hydroxyethyl and methoxyethyl-, wherein said methyl or ethyl is optionally substituted with 1, 2 or 3 fluoro substituents;
   each R⁹ is independently selected from H, methyl or ethyl;
   each R¹⁰ is independently selected from H and (C₁-C₄) alkyl wherein said (C₁-C₄) alkyl is optionally substituted by 1 or 2 substituents independently selected from methoxy, ethoxy, hydroxy and halo;
   or R⁹ and R¹⁰, together with the N atom to which they are attached, can join to form a saturated 5 or 6 membered heterocyclic ring further comprising ring carbon atoms and optionally one ring heteroatom independently selected from N, O and S, and wherein when the ring contains a S atom, said S is optionally substituted with one or two oxo substituents;
   R¹¹ is H, (C₁-C₄)alkyl, (C₁-C₄) alkoxy or halo;
   R¹² is H or halo;
   R¹³ is selected from NH₂, —C(O)OH, —NH(C(O)—CH₃) and —C(O)—NH(CH₃);
   R¹⁴ is selected from —C(O)—NR⁹(R¹⁰), (C₁-C₄)alkyl, —C(O)(C₁-C₄)alkyl, —C(O)O(C₁-C₄)alkyl;
   each R²³ is independently selected from H, halo, cyclopropyl and (C₁-C₄)alkyl;
   n is 1, 2 or 3;
   p is 0, 1, 2 or 3;

heterocyclyl¹ is a 3, 4, 5 or 6 membered fully saturated or partially unsaturated monocyclic group comprising ring carbon atoms and 1 or 2 ring heteroatoms independently selected from N, O and S;

heteroaryl² is 5 or 6 membered fully unsaturated monocyclic group comprising ring carbon atoms and 1, 2, 3 or 4 ring heteroatoms independently selected from N, O and S, wherein the total number of ring S atoms does not exceed 1, and the total number of ring O atoms does not exceed 1;

and
m is 0, 1 or 2.
* indicates the point of attachment to the remainder of the molecule. The compound of formula II can be prepared by the process disclosed in PCT/IB2013/050655. Further specific examples of the Mdm2 inhibitors are presented therein.

Particularly, the HDM-2/p53 inhibitor is (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one of formula (IV) (compound A, CGM097 or NVP-CGM097).

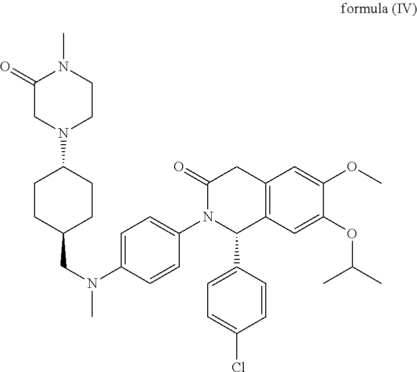 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

In one embodiment the HDM-2/p53 inhibitor is selected from the list of compounds as provided in the claims or items, or pharmaceutically acceptable salts thereof. In addition, it can be selected from the group consisting of:
(S)-1-(4-Chloro-phenyl)-7-isopropoxy-6-methoxy-2-(4-{methyl-[4-(3-oxo-piperazin-1-yl)-trans-cyclohexylmethyl]-amino}-phenyl)-1,4-dihydro-2H-isoquinolin-3-one,
(S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one,
(S)-1-(4-Chloro-phenyl)-7-isopropoxy-6-methoxy-2-(6-{methyl-[4-(4-methyl-3-oxo-piperazin-1-yl)-trans-cyclohexylmethyl]-amino}-pyridin-3-yl)-1,4-dihydro-2H-isoquinolin-3-one,
(S)-1-(4-Chloro-phenyl)-7-isopropoxy-6-methoxy-2-(6-{methyl-[4-(3-methyl-4-oxo-imidazolidin-1-yl)-trans-cyclohexylmethyl]-amino}-pyridin-3-yl)-1,4-dihydro-2H-isoquinolin-3-one,
(S)-1-(4-Chloro-phenyl)-7-isopropoxy-6-methoxy-2-(5-{methyl-[4-(3-methyl-4-oxo-imidazolidin-1-yl)-trans-cyclohexylmethyl]-amino}-pyrazin-2-yl)-1,4-dihydro-2H-isoquinolin-3-one,
1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one,
(S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one,
4-[(S)-5-(3-Chloro-2-fluoro-phenyl)-2-(2,4-dimethoxy-pyrimidin-5-yl)-3-isopropyl-6-oxo-3,4,5,6-tetrahydro-pyrrolo[3,4-d]imidazol-4-yl]-benzonitrile,
(S)-5-(5-Chloro-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one,
(S)-5-(3-chloro-4-fluorophenyl)-6-(4-chlorophenyl)-2-(2,4-dimethoxy-pyrimidin-5-yl)-1-((R)-1-methoxypropan-2-yl)-5,6-dihydropyrrolo[3,4-d]imidazol-4(1H)-one, and
(S)-5-(5-chloro-1-methyl-2-oxo-1,2-dihydropyridin-3-yl)-6-(4-chlorophenyl)-2-(2,4-dimethoxy-d6-pyrimidin-5-yl)-1-((R)-1-methoxypropan-2-yl)-5,6-dihydropyrrolo[3,4-d]imidazol-4(1H)-one.

In another embodiment the HDM-2/p53 inhibitor a compound selected from the group consisting of:

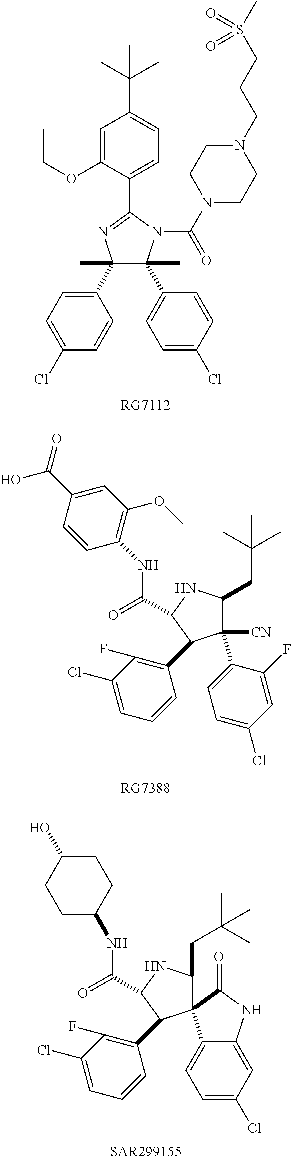

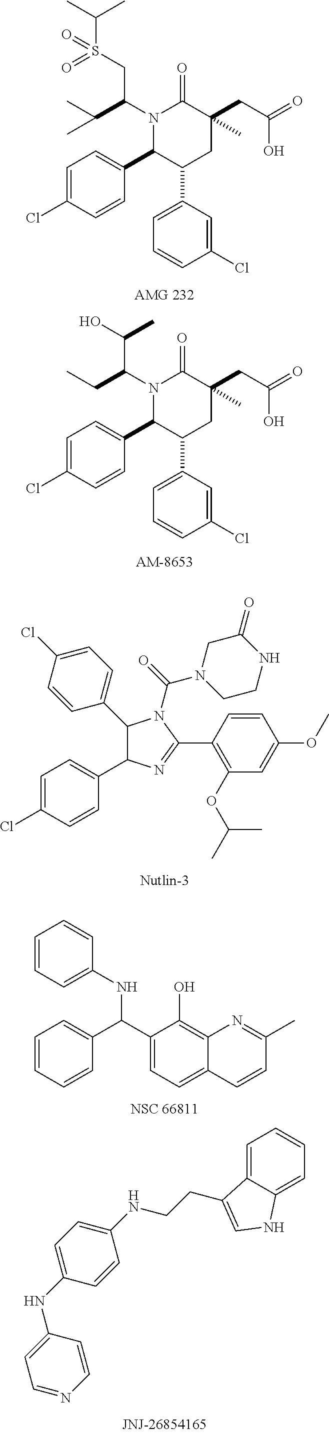

In another preferred embodiment, the HDM-2/p53 inhibitor is (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one.

In the present disclosure, the ALK inhibitor can be a compound that inhibits ALK with the IC50 of less than 100 μM, preferably less than 10 μM, more preferably less than 1 μM, measured by a Caliper mobility shift assay. The Caliper mobility shift technology is based on the separation of particles of different charges and sizes in an electrical field, similar to capillary electrophoresis. The Caliper kinase assays utilize fluorescently labeled peptides as kinase substrates. The phosphorylation of the peptide in the course of the reaction introduces additional negative charges via the phosphate and hence permits its separation from the phosphorylated peptide. Both, the separation and the detection of the labeled peptides take place in the microfluidic system of the Caliper Lab Chip. The LabChips have 12 “sippers” enabling the parallel analysis of 12 samples at the same time. The fact that both, unphosphorylated peptide (substrate) and phosphorylated peptide (product) are measured and that the separation makes the readout relatively insensitive to interference by fluorescent compounds results in the excellent data quality of this assay. General assay procedure can be performed at 30° C. for 60 min in a total volume of 9 μL including 0.050 μL of compound dilution or pure DMSO, respectively. The reaction can be terminated by the addition of 16 μL of stop solution (100 mM Hepes, 5% (v/v) DMSO, 0.1% (v/v) Coating reagent, 10 mM EDTA, 0.015% (v/v) Brij 35). After termination of the reactions, the plates are transferred into the Caliper LabChip 3000 workstation for analysis. The effect of a compound on the enzymatic activity is obtained from the linear progress curves in the absence and presence of the compound and routinely determined from one reading (end point measurement).

It can also be a compound of formula (III),

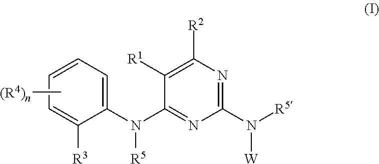

or pharmaceutically acceptable salts thereof; wherein

### W is

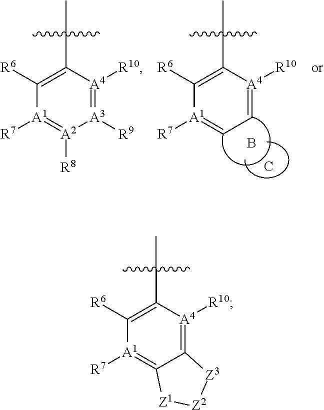

A¹ and A⁴ are independently C or N;
each A² and A³ is C, or one of A² and A³ is N when R⁶ and R⁷ form a ring;
B and C are independently an optionally substituted 5-7 membered carbocyclic ring, aryl, heteroaryl or heterocyclic ring containing N, —NH, O or S;
Z¹, Z² and Z³ are independently NR¹¹, C═O, CR—OR, (CR₂)₁₋₂ or ═C—R¹²;
R¹ and R² are independently halo, OR¹², NR(R¹²), SR¹², or an optionally substituted C₁₋₆ alkyl, C₂₋₆ alkenyl or C₂₋₆ alkynyl; or one of R¹ and R² is H;
R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;
R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;
R, R⁵ and R^{5′} are independently H or C₁₋₆ alkyl;
R⁸ and R⁹ are independently C₁₋₆ alkyl, C₂₋₆ alkenyl, C₂₋₆ alkynyl, halo or X, or one of R⁸ and R⁹ is H when R¹ and R² form a ring; and provided one of R⁸ and R⁹ is X;
alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, —NH, —NR¹O and/or S; or R⁷, R⁸, R⁹ and R¹⁰ are absent when attached to N;
R¹¹ is H, C₁₋₆ alkyl, C₂₋₆ alkenyl, (CR₂)ₚCO₁₋₂R, (CR₂)ₚOR, (CR₂)ₚR¹³, (CR₂)ₚNRR¹², (CR₂)ₚCONRR¹² or (CR₂)ₚSO₁₋₂R¹²;
R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;
X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;
Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)_{q}Y is 0.

In the above Formula (1), R¹ may be halo or C_{I-6} alkyl; R² is H or NH₂; or R^{I} and R² together form an optionally substituted 5-6 membered aryl, or heteroaryl or heterocyclic ring comprising 1-3 nitrogen atoms, In other examples, R³ in Formula (1) may be SO₂R¹², SO₂NH₂, SO₂NRR¹², CO₂NH₂, CONRR¹², CO_{I-2}R¹², or cyano; and R^{I2} is C_{I-6} alkyl, an optionally substituted C₃₋₇ cycloalkyl, C₃₋₇ cycloalkenyl, pyrrolidinyl, piperazinyl, piperidinyl (including but not limited to piperdin-4-yl and other related structural and positional isomers), morpholinyl or azetidinyl In yet other examples, R⁵, R^{5′}, R⁷ and R¹⁰ in Formula (1) are independently H, and n is 0, In other examples, R⁶ in Formula (I) may be halo or OR¹², and R^{I2} is C₁₋₆ alkyl.

In one embodiment, the ALK inhibitor compound of Formula (V) is

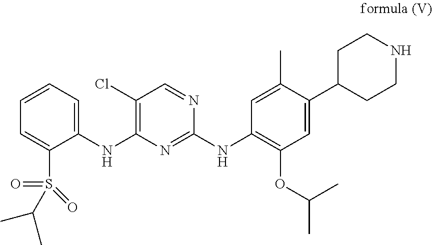 [SMILES: Cc1cc(Nc2ncc(Cl)c(Nc3ccccc3S(=O)(=O)C(C)C)n2)c(OC(C)C)cc1C1CCNCC1]

Also known by the IUPAC name 5-chloro-N2-(2-isopropoxy-5-methyl-4-(piperidin-4-yl)phenyl)-N4-[2-(propane-2-sulfonyl)-phenyl]-pyrimidine-2,4-diamine or pharmaceutically acceptable salts thereof.

In another embodiment, the ALK inhibitor can be a compound selected from the group consisting of:

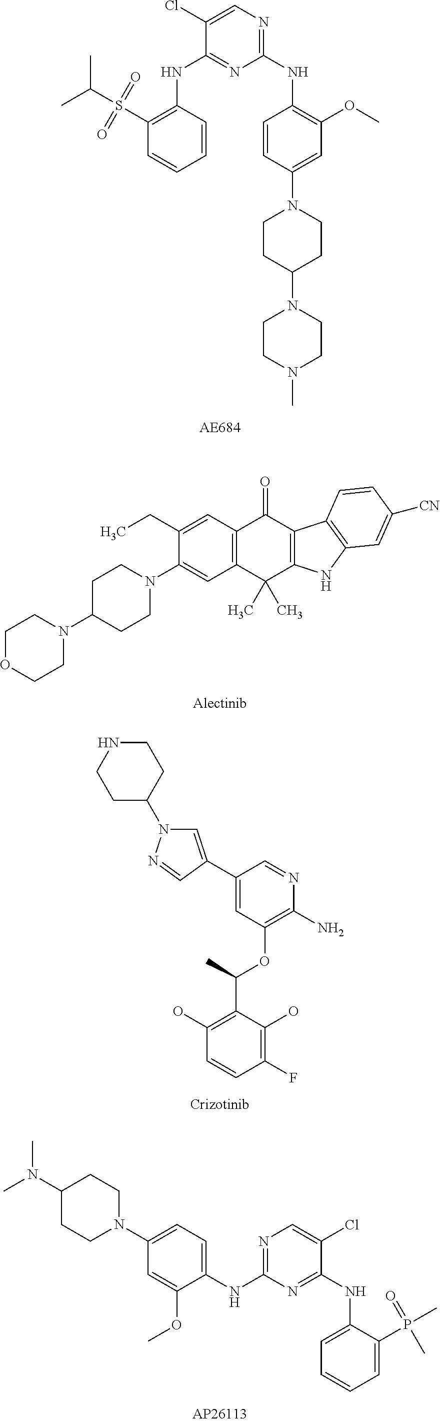

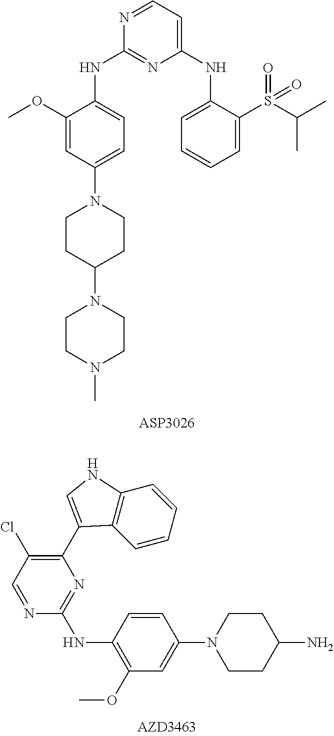 [SMILES: COc1cc(N2CCC(N3CCN(C)CC3)CC2)ccc1Nc1nccc(Nc2ccccc2S(=O)(=O)C(C)C)n1 ; COc1cc(N2CCC(N)CC2)ccc1Nc1ncc(Cl)c(-c2c[nH]c3ccccc23)n1]

Among HDM-2/p53 inhibitors and related mouse murine double minute two homolog inhibitors (also referred to as “mdm-2” or also known as “E3 ubiquitin-protein ligase HDM-2/p53 inhibitors) useful according to the disclosure, are selected from the group comprising NVP-CGM097; Caylin-1, Caylin-2, HLI373, Nutlin-3; SC204072 or a pharmaceutically acceptable salt thereof.

In one embodiment of the disclosure, a pharmaceutical combination of an effective amount of a ALK inhibitor, LDK378 (ceritinib) i.e. 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and at least one HDM-2/p53 inhibitor selected from the group comprising NVP-CGM097; Caylin-1, Caylin-2, HLI373, Nutlin-3; SC204072 or a pharmaceutically acceptable salt thereof; or at least one BRaf inhibitor selected from LGX818 for example, results in unexpected improvement in the treatment of cancer, including but not limited to neuroblastomas, metastatic neuroblastomas, mutant neuroblastomas, melanomas, metastatic melanomas, and mutant melanomas.

According to one embodiment, one HDMA-2/p53 inhibitor (or related Mdm-2 inhibitor) useful employed in accordance with the invented combination of an ALK inhibitor, for example LDK378, is 8-(2,6-Difluoro-3,5-dimethoxy-phenyl)-quinoxaline-5-carboxylic acid (4-dimethylaminomethyl-1H-imidazol-2-yl)-amide with the following chemical formula:

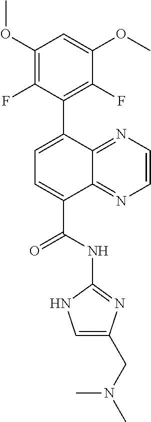 [SMILES: COc1cc(OC)c(F)c(-c2ccc(C(=O)Nc3nc(CN(C)C)c[nH]3)c3nccnc23)c1F]

Example 127 of WO 2009/141386 discloses compound structure as well as the method of making it. Compound is a small molecular mass inhibitor that is highly selective for FGFR1-4 in two t(4; 14) multiple myeloma cell lines, KMS-11 and OPM-2, harboring gain-of-function mutation, FGFR3-Y373C and FGFR3-K650E, respectively.

BRaf inhibitor according to the disclosure can be a compound selected for example from the group consisting of: (S)-methyl-1-(4-(3-(5-chloro-2-fluoro-3-(methylsulfonamido)phenyl)-1-isopropyl-1H-pyrazol-4-yl)pyrimidin-2-ylamino)propan-2-ylcarbamate;
methyl N-[(2S)-1-({4-[3-(5-chloro-2-fluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(2,5-difluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(5-chloro-2-fluoro-3-methanesulfonamidophenyl)-1-ethyl-1H-pyrazol-4-yl]pyrimidin-2-yl}amino)propan-2-yl]carbamate;
methyl N-[(2S)-1-({4-[3-(2-fluoro-3-methanesulfonamido-5-methylphenyl)-1-(propan-2-yl)-1H-pyrazol-4-yl]pyrimidin-2-yl}amino)propan-2-yl]carbamate;
methyl N-[(2S)-1-({4-[3-(2-chloro-3-methanesulfonamido-5-methylphenyl)-1-(propan-2-yl)-1H-pyrazol-4-yl]pyrimidin-2-yl}amino)propan-2-yl]carbamate;
methyl N-[(2S)-1-({4-[3-(2-chloro-5-fluoro-3-methanesulfonamidophenyl)-1-(propan-2-yl)-1H-pyrazol-4-yl]pyrimidin-2-yl}amino)propan-2-yl]carbamate;
methyl N-[(2R)-1-({4-[3-(5-chloro-2-fluoro-3-methanesulfonamidophenyl)-1-(propan-2-yl)-1H-pyrazol-4-yl]pyrimidin-2-yl}amino)propan-2-yl]carbamate;
methyl N-[(2S)-1-({4-[3-(2,5-dichloro-3-methanesulfonamidophenyl)-1-(propan-2-yl)-1H-pyrazol-4-yl]pyrimidin-2-yl}amino)propan-2-yl]carbamate; and
vemurafenib, or pharmaceutically acceptable salts thereof.

More specifically, the BRaf inhibitor can be either (S)-methyl-1-(4-(3-(5-chloro-2-fluoro-3-(methylsulfonamido)phenyl)-1-isopropyl-1H-pyrazol-4-yl)pyrimidin-2-ylamino)propan-2-ylcarbamate, methyl N-[(2S)-1-({4-[3-(5-chloro-2-fluoro-3-methanesulfonamidophenyl)-1-(propan-2-yl)-1H-pyrazol-4-yl]pyrimidin-2-yl}amino)propan-2-yl]carbamate or vemurafenib, or pharmaceutically acceptable salts thereof.

In a specific embodiment, the BRaf inhibitor is (S)-methyl-1-(4-(3-(5-chloro-2-fluoro-3-(methylsulfonamido)phenyl)-1-isopropyl-1H-pyrazol-4-yl)pyrimidin-2-ylamino)propan-2-ylcarbamate (LGX818), or a pharmaceutically acceptable salt thereof. It has a structure of formula (VI):

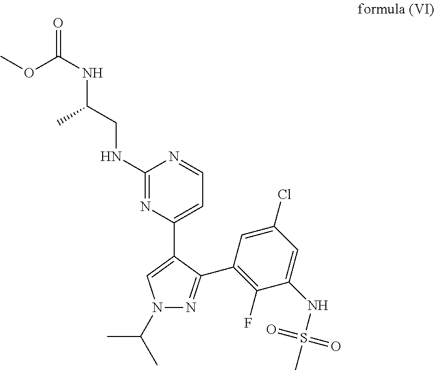 [SMILES: COC(=O)N[C@@H](C)CNc1nccc(-c2cn(C(C)C)nc2-c2cc(Cl)cc(NS(C)(=O)=O)c2F)n1]

The present disclosure further relates to a combined preparation or a pharmaceutical composition comprising (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier.

In one embodiment, the present disclosure relates to a combined preparation which comprises: (i) one or more unit dosage forms of combination partner (a), and (ii) one or more unit dosage forms of combination partner (b). The present disclosure particularly pertains to a pharmaceutical combination comprising (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier useful for treating or preventing a proliferative disease in a subject in need thereof.

The present disclosure also pertains to a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition or medicament for the treatment or prevention of a proliferative disease in a subject in need thereof.

The present disclosure also pertains to a pharmaceutical combination comprising: (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof, and optionally at least one pharmaceutically acceptable carrier useful for treating or preventing a proliferative disease in a subject in need thereof.

The present disclosure further pertains to the use of (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof for the preparation of a pharmaceutical composition or medicament for the treatment or prevention of a proliferative disease.

The present disclosure relates to a method of treating a subject having a proliferative disease, namely cancer, comprising the step of administering to said subject a pharmaceutical combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier in a quantity, which is jointly therapeutically effective against the proliferative disease or cancer.

The present disclosure further provides a commercial package comprising as therapeutic agents a combination comprising: (a) an anaplastic lymphoma kinase (ALK) inhibitor, or a pharmaceutically acceptable salt thereof, and (b) at least one HDM-2/p53 (or related HDM-2/p53) inhibitor or at least one BRaf inhibitor, or a pharmaceutically acceptable salt thereof, and optionally at least one pharmaceutically acceptable carrier for use in the preparation of a pharmaceutical composition, together with instructions for simultaneous, separate or sequential administration thereof for use in the delay of progression or treatment of a proliferative disease.

The above combinations are also provided for simultaneous, separate or sequential administration, in particular for treating or preventing a proliferative disease.

The combination of the two compounds according to the present disclosure, optionally comprising another chemotherapeutic agent, can be used for the treatment of proliferation disease or cancer. The nature of proliferative diseases is multifactorial. Under certain circumstances, drugs with different mechanisms of action may be combined. However, just considering any combination of therapeutic agents having different mode of action does not necessarily lead to combinations with advantageous effects. The administration of a pharmaceutical combination of the disclosure may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically therapeutic agents used in the combination of the disclosure. A further benefit is that lower doses of the therapeutic agents of the combination of the disclosure can be used, for example, that the dosages need not only often be smaller, but are also applied less frequently, or can be used in order to diminish the incidence of side-effects observed with one of the combination partners alone. This is in accordance with the desires and requirements of the patients to be treated.

The combination partners (i) and (ii) in any disclosure embodiment are preferably formulated or used to be jointly (prophylactically or especially therapeutically) active. This means in particular that there is at least one beneficial effect, e.g. a mutual enhancing of the effect of the combination partners (i) and (ii), in particular a synergism, e.g. a more than additive effect, additional advantageous effects (e.g. a further therapeutic effect not found for any of the single compounds), less side effects, a combined therapeutic effect in a non-effective dosage of one or both of the combination partners (i) and (ii), and very preferably a clear synergism of the combination partners (i) and (ii). For example, the compounds may be given separately or sequentially (in a chronically staggered manner, especially a sequence-specific manner) in such time intervals that they preferably, in the warm-blooded animal, especially human, to be treated, and still show a (preferably synergistic) interaction (joint therapeutic effect). A joint therapeutic effect can, inter alia, be determined by following the blood levels, showing that both compounds are present in the blood of the human to be treated at least during certain time intervals, but this is not to exclude the case where the compounds are jointly active although they are not present in blood simultaneously.

In a one embodiment of the present disclosure, the combination of the present disclosure can be used to treat proliferative disease is cancer. The cancer can be in principle any cancer that comprises mutated anaplastic lymphoma kinase (ALK). This means that any genetic change that leads to activation or higher activity of ALK compared to the activity of the ALK in healthy control is suitable for the treatment with the combination of the present disclosure. Mutated anaplastic lymphoma kinase (ALK) particularly refers to ALK comprising activating mutations such as, but not limited to, point mutations resulting in amino acid changes of F1174L, R1275Q, F1174C, F1245V, F1174V, D1091N, I1171N, F1174I, L1196M or F1245C, or amplification and translocation mutation including EML4-ALK or NPM1-ALK. The cancers harboring said mutations can be for example neuroblastoma, lung cancer or melanoma.

In one embodiment the cancer is neuroblastoma. The cancer can even be relapsed or high-risk neuroblastoma. Relapsed neuroblastoma means that the patient has already been treated with adequate treatment, be it an ALK inhibitor alone, a HDM-2/p53 inhibitor alone, or another chemotherapeutic agent, but the cancer appeared again or progressed. High-risk neuroblastoma means neuroblastoma of:
Stage 2A or 2B disease and MYCN amplification
Stage 3 disease and MYCN amplification
Stage 3 disease in children age 18 months or older, no MYCN amplification, and unfavorable histopathology
Stage 4 disease in children younger than 12 months and MYCN amplification
Stage 4 disease in children between 12 months and 18 months old with MYCN amplification, and/or diploidy, and/or unfavorable histology
Stage 4 disease in children 18 months or older
Stage 4S disease and MYCN amplification, wherein stages 2 to 4S are classified based on the International Neuroblastoma Staging System Committee (INSS) system.

In one embodiment, the combination is used to treat a pediatric patient, i.e. a patient of age below 20 years. The age of a pediatric patient, where childhood cancer (also known as pediatric cancer) is treated, can be 0-14 years inclusive, that is, up to 14 years and 11.9 months of age. The age of pediatric patient and/or childhood cancer can also include young adults between 15-19 years old.

In another embodiment the cancer is lung cancer.

In yet another embodiment, the cancer is melanoma.

Best treatment results are obtained in cancer with functional p53 or p53 wt.

In one embodiment, it has been discovered that the combination therapy comprising the combination of the disclosure results in unexpected improvement in the treatment or prevention of proliferative diseases as compared to the monotherapy with LDK378, crizotinib or patients who are resistant to crizotinib. When administered simultaneously, sequentially or separately, the ALK inhibitor and the HDMA-2/p53 receptor inhibitor interact synergistically to inhibit cell proliferation.

The present disclosure thus pertains to a combination product for simultaneous or sequential use, such as a combined preparation or a pharmaceutical fixed combination, or a combination of such preparation and combination.

In the combination therapies of the disclosure, the compounds useful according to the disclosure may be manufactured and/or formulated by the same or different manufacturers. Moreover, the combination partners may be brought together into a combination therapy: (i) prior to release of the combination product to physicians (e.g. in the case of a kit comprising the compound of the disclosure and the other therapeutic agent); (ii) by the physician themselves (or under the guidance of a physician) shortly before administration; (iii) in the patient themselves, e.g. during sequential administration of the compound of the disclosure and the other therapeutic agent.

In one embodiment, a data carrier comprising information about using (i) a HDM-2/p53 inhibitor or a pharmaceutically acceptable salt thereof, and (ii) an ALK inhibitor, or about using an ALK inhibitor and Braf inhibitor, in each case simultaneously or sequentially, is provided. The data carrier, for example in a form of a product information leaflet or a label, packaging, brochure or web page instruction can be used to instruct to administer (i) a HDM-2/p53 inhibitor of formula I or formula II, or a pharmaceutically acceptable salt thereof, and (ii) a BRAF inhibitor, or a pharmaceutically acceptable salt thereof, simultaneously or sequentially for the treatment of cancer. The data carrier is particularly useful in the event the two partners of the combination are not formulated together, and supplied or sold separately. Each of the partners can be supplied with the data carrier, or even have the data carrier detached or provided separately, that informs or instructs about the possibility to use the combination partner in a pharmaceutical combination of the present disclosure. The data carrier can be used for the same purpose also in fixed combinations or situations, where both partners are supplied or sold together.

In certain embodiment, any of the above pharmaceutical combination, use, administration, composition, method, product or formulation involve further administering one or more other (e.g. third) co-agents, especially a chemotherapeutic agent.

Thus, the disclosure relates in a further embodiment to a pharmaceutical combination, particularly a pharmaceutical composition or a product comprising a therapeutically effective amount of (i) a HDM-2/p53 inhibitor and (ii) an ALK inhibitor, or of (a) ALK inhibitor and (b) Braf inhibitor, or a pharmaceutically acceptable salt thereof, respectively, and at least one third therapeutically active agent (co-agent). The additional co-agent is preferably selected from the group consisting of an anti-cancer agent and an anti-inflammatory agent, particularly is an anti-cancer agent. Also in this case, the combination partners forming a corresponding combination according to the disclosure may be mixed to form a fixed pharmaceutical composition or they may be administered separately or pairwise (i.e. before, simultaneously with or after the other drug substance(s)).

A possible co-agent that can be added to the combination of the present disclosure is of the structure selected from the group consisting of:

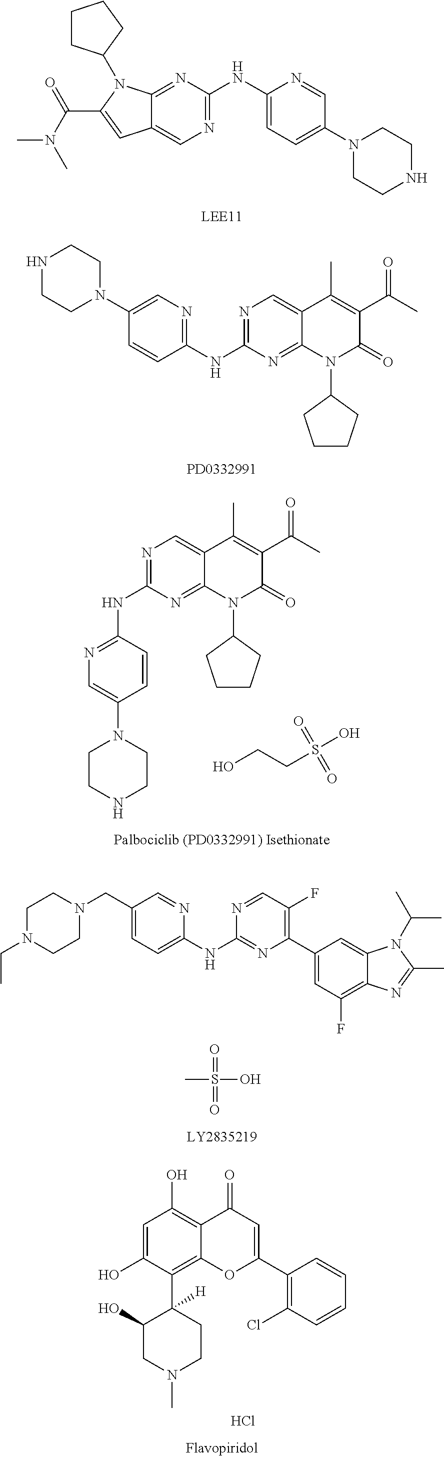

A combination product according to the disclosure can besides or in addition be administered especially for cancer therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemo-preventive therapy, for example in patients at risk.

Possible anti-cancer agents (e.g. for chemotherapy) as co-agents include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity; anti-angiogenic compounds; compounds which target, decrease or inhibit the activity of a protein or lipid phosphatase; gonadorelin agonists; anti-androgens; methionine amino peptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors; kinesin spindle protein inhibitors; MEK inhibitors; leucovorin; EDG binders; antileukemia compounds; ribonucleotide reductase inhibitors; S-adenosylmethionine decarboxylase inhibitors; angiostatic steroids; corticosteroids; other chemotherapeutic compounds (as defined below); photosensitizing compounds.

Further, alternatively or in addition combination products according to the disclosure may be used in combination with other tumor treatment approaches, including surgery, ionizing radiation, photodynamic therapy, implants, e.g. with corticosteroids, hormones, or they may be used as radiosensitizers.

It can be shown by established test models that the combination of the disclosure results in the beneficial effects described herein before. The person skilled in the art is fully enabled to select a relevant test model to prove such beneficial effects. The pharmacological activity of a combination of the disclosure may, for example, be demonstrated in a clinical study or in a test procedure as essentially described hereinafter.

Suitable clinical studies are in particular, for example, open label, dose escalation studies in patients with a proliferative disease. Such studies prove in particular the synergism of the therapeutic agents of the combination of the disclosure. The beneficial effects on proliferative diseases may be determined directly through the results of these studies which are known as such to a person skilled in the art. Such studies may be, in particular, be suitable to compare the effects of a monotherapy using either therapeutic agent and a combination of the disclosure.

Determining a synergistic interaction between one or more components, the optimum range for the effect and absolute dose ranges of each component for the effect may be definitively measured by administration of the components over different w/w ratio ranges and doses to patients in need of treatment.

In a separate embodiment, the present disclosure provides a synergistic combination for human administration comprising: (i) a HDM-2/p53 inhibitor and (ii) an ALK inhibitor, or a pharmaceutically acceptable salt thereof, respectively. Equally, the present disclosure provides a synergistic combination for human administration comprising (a) ALK inhibitor and (b) Braf inhibitor, or a pharmaceutically acceptable salt thereof, respectively. In particular embodiment, the synergistic combination comprises (a) 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof, and (b) at least one HDM-2/p53 inhibitor selected from comprising NVP-CGM097 or at least one BRaf inhibitor selected from (S)-methyl 1-(4-(3-(5-chloro-2-fluoro-3-(methylsulfonamido)phenyl)-1-isopropyl-1H-pyrazol-4-yl)pyrimidin-2-ylamino)propan-2-ylcarbamate or a pharmaceutically acceptable salt thereof. The combination can optionally further comprise at least one pharmaceutically acceptable carrier. The combination partners can be in a combination range (w/w) which corresponds to the ranges observed in a tumor model, e.g., as described in the Examples below, used to identify a synergistic interaction.

It is one objective of this disclosure to provide a pharmaceutical composition comprising a quantity, which is jointly therapeutically effective against a proliferative disease comprising the combination of the disclosure. In this composition, the combination partners (i) a HDM-2/p53 inhibitor and (ii) an ALK inhibitor, or (a) ALK inhibitor and (b) Braf inhibitor can be either administered in a single formulation or unit dosage form, administered concurrently, but optionally separately, or administered sequentially by any suitable route. The unit dosage form may also be a fixed combination.

The pharmaceutical compositions for separate administration of both combination partners, or for the administration in a fixed combination, i.e. a single galenical composition comprising the combination of the disclosure, may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers, especially suitable for enteral or parenteral application. The novel pharmaceutical composition contains may contain, from about 0.1% to about 99.9%, preferably from about 1% to about 60%, of the therapeutic agent(s). Suitable pharmaceutical compositions for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL™, available from Pfizer.

The unit dosage forms of the present disclosure may optionally further comprise additional conventional carriers or excipients used for pharmaceuticals. Examples of such carriers include, but are not limited to, disintegrants, binders, lubricants, glidants, stabilizers, and fillers, diluents, colorants, flavours and preservatives. One of ordinary skill in the art may select one or more of the aforementioned carriers with respect to the particular desired properties of the dosage form by routine experimentation and without any undue burden. The amount of each carriers used may vary within ranges conventional in the art. The following references which are all hereby incorporated by reference disclose techniques and excipients used to formulate oral dosage forms. See The Handbook of Pharmaceutical Excipients, 4^{th} edition, Rowe et al., Eds., American Pharmaceuticals Association (2003); and Remington: the Science and Practice of Pharmacy, 20^{th} edition, Gennaro, Ed., Lippincott Williams & Wilkins (2003). These optional additional conventional carriers may be incorporated into the oral dosage form either by incorporating the one or more conventional carriers into the initial mixture before or during granulation or by combining the one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE™ XL from International Specialty Products (Wayne, N.J.); cross-linked sodium carboxymethylcellulose or croscarmellose sodium, e.g., AC-DI-SOL™ from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL™ PH from FMC (Philadelphia, Pa.), hydroxypropyl cellulose hydroxylethyl cellulose and hydroxylpropylmethyl cellulose METHOCEL™ from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

In one embodiment, the present disclosure also pertains to a combination of the disclosure for use in the preparation of a pharmaceutical composition or medicament for the treatment or prevention of a proliferative disease in a subject in need thereof. In one embodiment, the proliferative disease is cancer, particularly neuroblastoma or melanoma.

In accordance with the present disclosure, a therapeutically effective amount of each of the combination partner of the combination of the disclosure may be administered simultaneously or sequentially and in any order, and the components may be administered separately or as a fixed combination. For example, the method of treating a proliferative disease according to the disclosure may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the disclosure may be administered separately at different times during the course of therapy or concurrently in divided or single combination forms. Furthermore, the term “administering” also encompasses the use of a pro-drug of a combination partner that convert in vivo to the combination partner as such. The instant disclosure is therefore to be understood as embracing all such regimens of simultaneous or alternating treatment and the term “administering” is to be interpreted accordingly.

The effective dosage of each of the combination partners employed in the combination of the disclosure may vary depending on the particular compound or pharmaceutical composition employed, the mode of administration, the condition being treated, and the severity of the condition being treated. Thus, the dosage regimen of the combination of the disclosure is selected in accordance with a variety of factors including the route of administration and the renal and hepatic function of the patient. A clinician or physician of ordinary skill can readily determine and prescribe the effective amount of the single therapeutic agents required to alleviate, counter or arrest the progress of the condition.

The optimum ratios, individual and combined dosages, and concentrations of the combination partners of the combination of the disclosure that yield efficacy without toxicity are based on the kinetics of the therapeutic agents' availability to target sites, and may be determined using methods known to those of skill in the art.

The effective dosage of each of the combination partners may require more frequent administration of one of the compound(s) as compared to the other compound(s) in the combination. Therefore, to permit appropriate dosing, packaged pharmaceutical products may contain one or more dosage forms that contain the combination of compounds, and one or more dosage forms that contain one of the combination of compounds, but not the other compound(s) of the combination.

When the combination partners, which are employed in the combination of the disclosure, are applied in the form as marketed as single drugs, their dosage and mode of administration can be in accordance with the information provided on the package insert of the respective marketed drug, if not mentioned herein otherwise.

The optimal dosage of each combination partner for treatment of a proliferative disease can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each combination partner that may be combined with the carrier materials to produce a single dosage form will vary depending upon the individual treated and the particular mode of administration. In some embodiments the unit dosage forms containing the combination of agents as described herein will contain the amounts of each agent of the combination that are typically administered when the agents are administered alone.

Frequency of dosage may vary depending on the compound used and the particular condition to be treated or prevented. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

The present disclosure further provides a commercial package comprising as therapeutic agents combination of the disclosure, together with instructions for simultaneous, separate or sequential administration thereof for use in the delay of progression or treatment of a proliferative disease in a subject in need thereof.

The combination product of the present disclosure is especially appropriate for treatment a patient suffering from a proliferative disorder, in particular a solid tumor, for example, melanoma, colorectal cancer, sarcoma, lung cancer, thyroid cancer and leukemia. The present disclosure further relates to a method of treating a subject having a proliferative disease comprising administered to said subject a combination of the disclosure in a quantity, which is jointly therapeutically effective against a neuroblastoma or a melanoma. In one embodiment the cancer that can be treated by the pharmaceutical combination is melanoma. In further embodiment, the cancer comprises BRAF having the V600E mutation. In yet another embodiment, the cancer comprises functional p53 or p53 wt.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to (1) at least partially alleviate, inhibit, prevent and/or ameliorate a condition, or a disorder or a disease (i) mediated by HDM-2/p53 and/or mediated by ALK activity, or (ii) characterized by activity (normal or abnormal) of HDM-2/p53 and/or BRAF; or (2) reduce or inhibit the activity of HDM-2/p53 and/or of BRAF; or (3) reduce or inhibit the expression of HDM-2/p53 and/or BRAF; or a disorder or a disease (i) mediated by HDM-2/p53 and/or mediated by ALK activity, or (ii) characterized by activity (normal or abnormal) of HDM-2/p53 and/or ALK; or to (2) reduce or inhibit the activity of HDM-2/p53 and/or of ALK; or to (3) reducing or inhibit the expression of HDM-2/p53 and/or ALK; or. A “subtherapeutic” dose as used herein describes the dose that does not lead to clinically satisfactory effect.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

The ALK inhibitor may be administered to a suitable subject daily in single or divided doses at an effective dosage in the range of about 0.05 to about 50 mg per kg body weight per day, preferably about 0.1-25 mg/kg/day, more preferably from about 0.5-10 mg/kg/day, in single or divided doses. For a 70 kg human, this would amount to a preferable dosage range of about 35-700 mg per day. Daily dose of LDK378 can be for example 750 mg.

HDM-2/p53 inhibitor of formula I or II of the pharmaceutical combination can be administered in unit dosage of about 1-5000 mg of active ingredient(s) for a subject of about 50-70 kg, or about 1 mg-3 g or about 1-250 mg or about 1-150 mg or about 0.5-100 mg, or about 1-50 mg of active ingredients. HDM-2/p53 inhibitor of formula I or II can be administered at a dose of between 200 mg and 1600 mg, particularly between 200 and 1200 mg daily. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

BRAF inhibitor of the present disclosure can be administered in therapeutically effective amounts via any of the usual and acceptable modes known in the art. A therapeutically effective amount may vary widely depending on the severity of the disease, the age and relative health of the subject, the potency of the compound used and other factors. In general, satisfactory results are indicated to be obtained systemically at daily dosages of from about 0.03 to 30 mg/kg per body weight. An indicated daily dosage in the larger mammal, e.g. humans, is in the range from about 0.5 mg to about 2000 mg, conveniently administered, e.g. in divided doses up to four times a day or in retard form. Suitable unit dosage forms for oral administration comprise from about 1 to 500 mg active ingredient.

In general, the dosage of the active ingredient to be applied to a warm-blooded animal depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical combination product according to the disclosure (as fixed combination, or as kit, e.g. as combination of a fixed combination and individual formulations for one or both combination partners or as kit of individual formulations of the combination partners) comprises the combination of the present disclosure and one or more pharmaceutically acceptable carrier materials (carriers, excipients). The pharmaceutical combination or the combination partners constituting it can be formulated for particular routes of administration such as oral administration, parenteral administration, and rectal administration, etc. In addition, the combination products of the present disclosure can be made up in a solid form (including without limitation capsules, tablets, pills, granules, powders or suppositories), or in a liquid form (including without limitation solutions, suspensions or emulsions). The combination products and/or their combination partners can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

In one embodiment, the pharmaceutical compositions are tablets or gelatin capsules comprising the active ingredient together with one or more commonly known carriers, e.g. one or more carriers selected from the group consisting of
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Tablets may be either film coated or enteric coated according to methods known in the art.

Suitable compositions for oral administration especially include an effective amount of one or more or in case of fixed combination formulations each of the combination partners (active ingredients) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient(s) in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Parenteral compositions, transdermal, topical compositions and other can be prepared by known methods in the art.

The following Examples illustrate the disclosure described above; they are not, however, intended to limit the scope of the disclosure in any way. The beneficial effects of the pharmaceutical combination of the present disclosure can also be determined by other test models known as such to the person skilled in the pertinent art.

### Example 1

### HDM-2/p53 Inhibitor and an Anaplastic Lymphoma Kinase (ALK) Inhibitor in Neuroblastoma

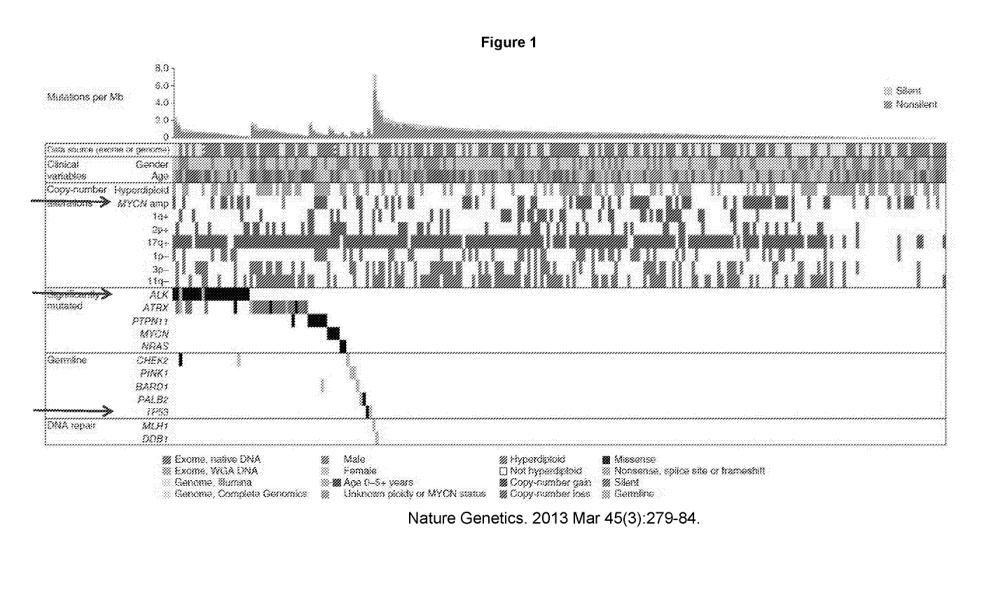

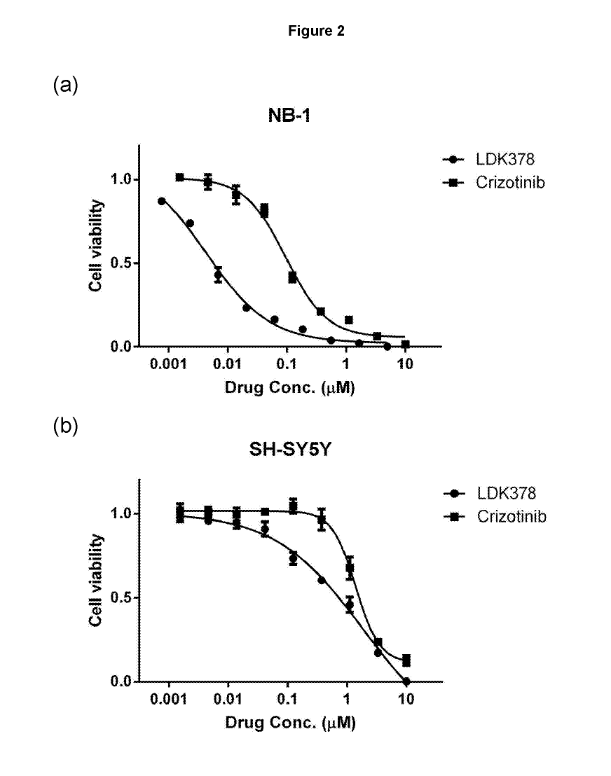

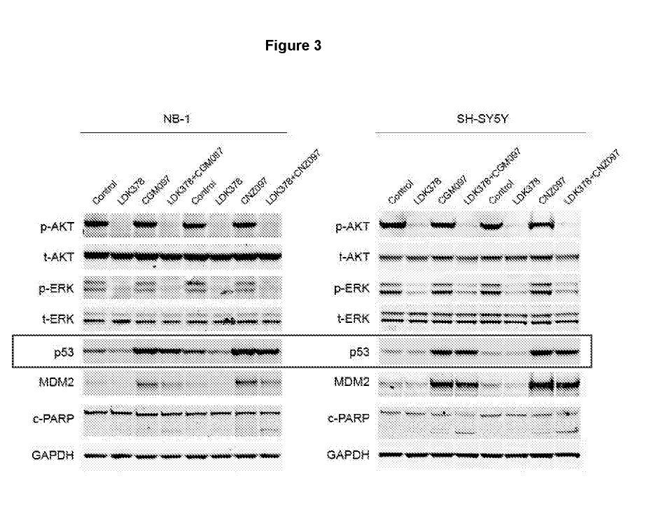

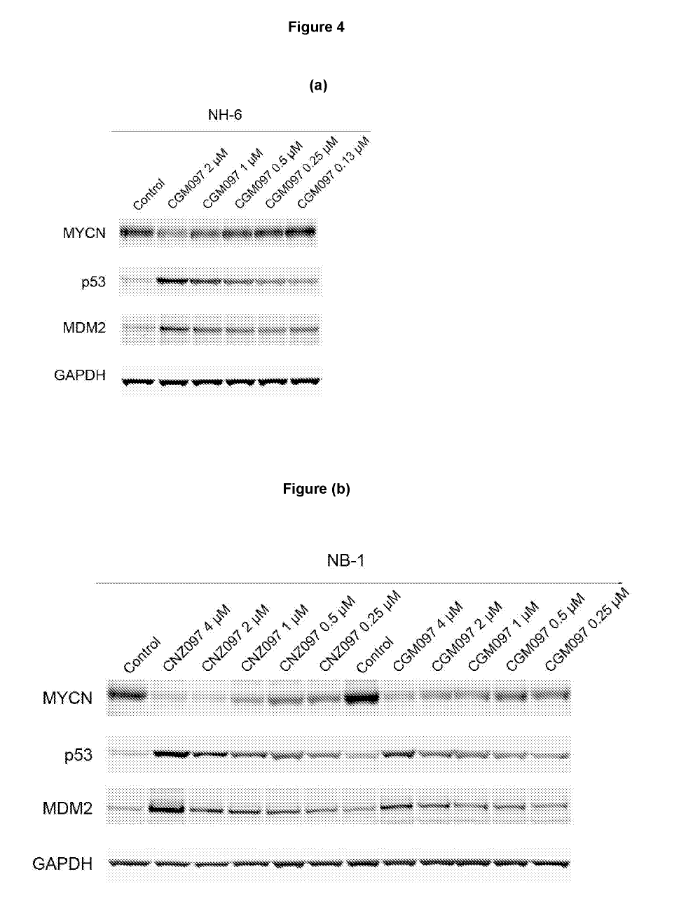

Neuroblastoma is the most common cancer in infancy, accounting for 15% of all childhood cancer-related death. MYCN amplification is the major genetic aberration in high-risk neuroblastoma and is associated with poor outcome (please refer to FIGS. 1 and 2). Genome-wide association studies have identified activation mutations and high-level amplification of ALK in approximately 10% of neuroblastoma patients (FIG. 3). In addition, ALK mutations can coexist with MYCN amplification, which defines a subset of ultra-high-risk neuroblastoma patients (FIG. 4). In contrast to the high frequency of p53 mutations observed in many human cancers of adults, mutations of p53 are less common in childhood cancers and have been reported in less than 2% of neuroblastomas. Wild-type (WT) p53 is required for the activation of p53 signaling by HDM-2/p53 inhibitors. This suggests that neuroblastoma could be amenable to intervention with HDM-2/p53 inhibitors. In this study (LDK378 and NVP-CGM097 in ALK+NB (p53 WT) cell lines), as a proof of concept, ALK inhibitor 5-chloro-N2-(2-isopropoxy-5-methyl-4-(piperidin-4-yl)phenyl)-N4-[2-(propane-2-sulfonyl)-phenyl]-pyrimidine-2,4-diamine (LDK378, compound B), in combination with a HDM-2/p53 inhibitor (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one (CGM097, Compound A) demonstrated that the combination promoted apoptosis in ALK mutant and p53 WT neuroblastoma cell lines.

### Materials and Methods

### Compound Preparation for In Vitro Experimentation

Compound stocks of LDK378 (compound B) and NVP-CGM097 (compound A) are prepared in DMSO at a final concentration of 10 mM. Working stocks of compound A and compound B are serially diluted in the appropriate cell culture medium to achieve final assay concentrations ranging from 10 μM to 0.039 μM.

### Cell Lines and Cell Culture

ALK+NB neuroblastoma cell line is disclosed in G. Barone, et al in the journal Clin. Cancer Res., Vol 19, pp 5814-5821 (2013).

### Cell Proliferation in Combination Dose Matrix

Cells are seeded at a selected density of 1000-7000 cells per 100 μl of medium per well in 96-well plates and incubated overnight prior to compound addition. Compound stock is freshly prepared in the appropriate culture medium and manually added to the plates by electronic multichannel pipette in three replicates. Cells were treated with LDK378 alone or with a combination of compound A and compound B diluted 1:2 for a ten point dilution ranging from 0.039 μM to 10 μM. The viability of cells is assessed at the time of compound addition and after a pre-determined hours of treatment by quantification of cellular level parameters according to the ALK+NB cell line protocol. Plates are read on a luminescence plate reader (Victor X4, Perkin Elmer). Fractional inhibition of growth is calculated using XLfit and normalized to no compound wells. For growth inhibition, day 0 values are subtracted before calculating inhibition. Data is analyzed by Chalice software (http://chalice.zalicus.com/documentation/analyzer/index.jsp) to calculate growth inhibition, inhibition and HSA excess using methods known in the art.

### Results Summary

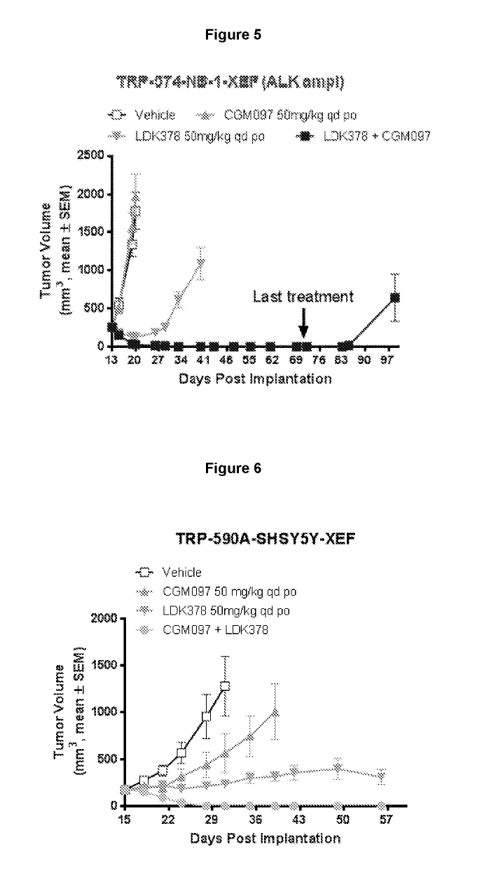

LDK378 inhibited ALK phosphorylation and CGM097 caused induction of p53 and its downstream target genes in these cell lines (FIG. 5). FIG. 5 summarizes data for LDK378 Single Agent and Combination Treatments with CGM097 in a NB-1 Xenograft. LDK378 and CGM097 combination resulted in complete tumor regression. Tumors resumed growth after treatment termination. Similar results were obtained with the cell line TRP-590A-SHSYSY-XEF (ALK F1174L mutated) (FIG. 6). Under continuous treatment, tumors in LDK378 single agent treated group and CGM097 treated group continued to grow. Tumors in LDK378+CGM097 treated group remained small under treatment.

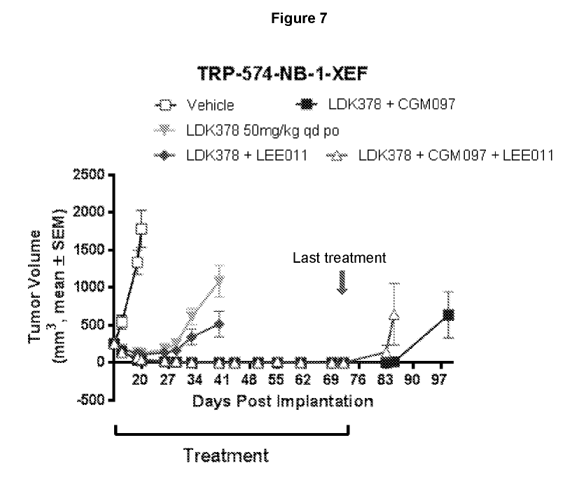

FIG. 7 shows that under continuous treatment, tumors in LDK378 single agent treated group and LDK378+LEE011 treated group resumed growth before day 41. Tumors in LDK378+CGM097 treated group and LDK378+CGM097+LEE011 treated group remained small under treatment. After treatment termination, tumors resumed growth. Meanwhile, HDM-2/p53 inhibition in MYCN-amplified neuroblastoma cell lines significantly decreased the levels of Mycn protein. In addition, LDK378 and CGM097 combination resulted in complete tumor regression and markedly prolonged survival in neuroblastoma xenograft models. Overall, LDK378 and CGM097 combination may provide an effective treatment for ALK mutant and p53 WT neuroblastoma patients.

### Example 2

### HDM-2/p53 Inhibitor and an Anaplastic Lymphoma Kinase (ALK) Inhibitor in Neuroblastoma In Vivo

The combination of LDK378 (Compound B) and HDM-2/p53 inhibitor (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one (Compound C) were tested in NB-1 neuroblastoma in vivo xenograft model. A total of 5 animals per group were enrolled in efficacy study. For single-agent and combination studies, animals were dosed via oral gavage for both LDK378 and Compound C. LDK378 was formulated in 0.5% CMC/0.5% Tween 80, and Compound C was formulated in Methylcellulose 0.5% w/V in pH 6.8 50 mM phosphate buffer at 20 mg/kg as free base. For NB1 model, the tumors reached approximately 200 mm³ at day 16 post implantation. On Day 16, tumor-bearing mice were randomized into treatment groups.

The design of the study including dose schedule for all treatment groups are summarized in the Table 1. Animals were weighed at dosing day(s) and dose was body weight adjusted, dosing volume was 10 ml/kg. Tumor dimensions and body weights were collected at the time of randomization and twice weekly thereafter for the study duration. The following data was provided after each day of data collection: incidence of mortality, individual and group average body weights, and individual and group average tumor volume.

TABLE 1
Study design

| | | | | Number of |
|---|---|---|---|---|
| Groups | Treatment | Dose | Schedule | mice |
| 1 | Vehicle | 0.5% CMC/0.5% Tween | PO QD | 5 |
| | | 80 | | |
| | | 0.5% MC in phosphate | PO QD | |
| | | buffer | | |
| 2 | LDK378 | 50 mg/kg | PO QD | 5 |
| 3 | Compound C | 20 mg/kg | PO QD | 5 |
| 4 | LDK378 | 50 mg/kg | PO QD | 5 |
| | Compound C | 20 mg/kg | PO QD | |

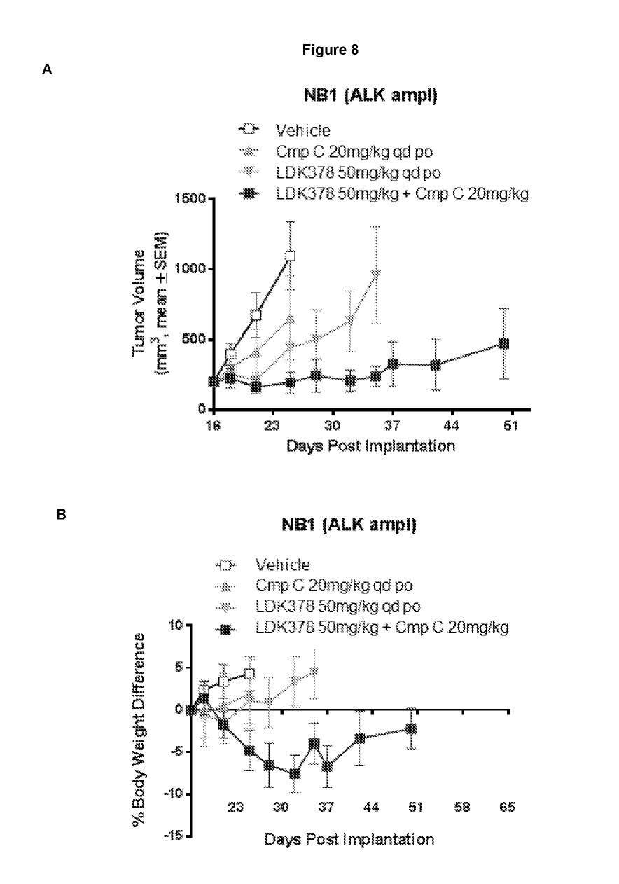

FIG. 8 summarizes data for LDK378 single agent and combination treatments with Compound C in NB-1 xenograft. On day 25, Compound C resulted in T/C of 50.8%, LDK378 exhibited T/C of 27.6%, combination of LDK378 with Compound C resulted in tumor stasis with T/T0 as −3.4%, which is statistically significant compared to vehicle treated group, but not Compound C or LDK378 monotherapy groups (Table 2). Tumors exhibited continued growth under treatment. Tumors in LDK378+Compound C treated groups grow relative slow compared to LDK378 monotherapy. Combination of LDK378 with Compound C resulted in maximum body weight loss of −7.6% on day 32, afterwards, mouse body weight started to recover. Overall, LDK378 and Compound C combination may provide an effective treatment for neuroblastoma patients, particularly ALK amplified and p53 WT neuroblastoma patients.

TABLE 2
Anti-tumor effects of Compound C, LDK378 and combination
of Compound C with LDK378 in NB1 model

| | T/C % on day 25 | T/T0 % on day 25 |
|---|---|---|
| Vehicle | 100% | |
| COMPOUND C 20 mg/kg qd po | 50.80% | |
| LDK378 50 mg/kg qd po | 27.60% | |
| LDK378 50 mg/kg + | | −3.40%* |
| COMPOUND C 20 mg/kg | | |

*p < 0.05 by one way ANOVA followed by Tukey test.

1.-42. (canceled)

43. A pharmaceutical combination comprising (i) a HDM-2/p53 inhibitor (S)-1-(4-Chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, or a pharmaceutically acceptable salt thereof, or (S)-5-(5-Chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one or a pharmaceutically acceptable salt thereof; and (ii) an anaplastic lymphoma kinase (ALK) inhibitor 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof.

44. The pharmaceutical combination according to claim 43, wherein the pharmaceutical combination comprises (i) a HDM-2/p53 inhibitor, or a pharmaceutically acceptable salt thereof, and (ii) an anaplastic lymphoma kinase (ALK) inhibitor, or a pharmaceutically acceptable salt thereof, separately or together.

45. The pharmaceutical combination according to claim 43 for simultaneous or sequential use of the (i) a HDM-2/p53 inhibitor, or a pharmaceutically acceptable salt thereof, and (ii) an anaplastic lymphoma kinase (ALK) inhibitor, or a pharmaceutically acceptable salt thereof.

46. The pharmaceutical combination according to claim 43, further comprising at least one pharmaceutically acceptable carrier.

47. The pharmaceutical combination according claim 43 in the form of a fixed combination.

48. The pharmaceutical combination according to claim 43 in the form of a kit of parts for the combined administration, wherein the HDM-2/p53 inhibitor, or a pharmaceutically acceptable salt thereof, and the anaplastic lymphoma kinase (ALK) inhibitor, or a pharmaceutically acceptable salt thereof, are administered jointly or independently at the same time or separately within time intervals.

49. The pharmaceutical combination according to claim 43 in the form of a pharmaceutical composition.

50. The pharmaceutical combination according to claim 43, wherein (i) a HDM-2/p53 inhibitor, or a pharmaceutically acceptable salt thereof, and (ii) an anaplastic lymphoma kinase (ALK) inhibitor, or a pharmaceutically acceptable salt thereof, are in a quantity which is jointly therapeutically effective for the treatment of cancer.

51. The pharmaceutical combination according to claim 43 in the form of a combination product or a pharmaceutical composition.

52. A method of treating cancer in a patient comprising administering simultaneously or sequentially a therapeutically effective amount of (i) a HDM-2/p53 inhibitor, or a pharmaceutically acceptable salt thereof, and (ii) an anaplastic lymphoma kinase (ALK) inhibitor, or a pharmaceutically acceptable salt thereof.

53. The method of treating cancer in a patient according to claim 52, wherein the cancer comprises mutated anaplastic lymphoma kinase (ALK).

54. The method of treating cancer in a patient according to claim 52, wherein the cancer is neuroblastoma.

55. The method of treating cancer in a patient according to claim 52, wherein the cancer is relapsed or high-risk neuroblastoma.

56. The method of treating cancer in a patient according to claim 52, wherein the cancer comprises functional p53 or p53 wt.

57. The pharmaceutical combination according to claim 43 for the manufacture of a medicament or a pharmaceutical product for the treatment of cancer.
